(19) United States
(12) Patent Application Publication
(10) Pub. No.: US 2026/0048031 A1
(43) Pub. Date: Feb. 19, 2026

(54) POLYMORPHS OF CO-CRYSTALS OF ITANAPRACED AND NICOTINAMIDE

(71) Applicant: CereSpir Incorporated, New York, NY (US)

(72) Inventors: Daniel Chain, New York, NY (US); Matt Forster, Northbrook, IL (US); Richard Margolin, Cambridge, MA (US); Ronald Mueller, Northbrook, IL (US)

(21) Appl. No.: 19/210,006

(22) Filed: May 16, 2025

Related U.S. Application Data
(60) Provisional application No. 63/649,341, filed on May 18, 2024.

(51) Int. Cl. A61K 31/192; A61K 9/14; A61K 31/455; C07C 61/40; C07D 213/82

(52) U.S. Cl. CPC A61K 31/192; A61K 9/141; A61K 31/455; C07C 61/40; C07D 213/82; C07B 2200/13; C07C 2601/02

(57) ABSTRACT

Polymorphs of co-crystals of itanapraced and nicotinamide and pharmaceutical compositions and dosage forms containing the polymorphs are described. Methods of synthesizing the polymorphs and uses of polymorphs are also described.

### FIELD OF THE INVENTION

The invention generally relates to polymorphs of co-crystals of itanapraced and nicotinamide; methods of preparation of the polymorphs; pharmaceutical compositions comprising the polymorphs; methods of preparation of pharmaceutical compositions comprising the polymorphs; pharmaceutical dosage forms comprising the polymorphs; uses of the polymorphs as active pharmaceutical ingredients (APIs) in pharmaceutical dosage forms; methods of preparation of pharmaceutical dosage forms comprising the polymorphs; and uses of the polymorphs in preparation of pharmaceutical dosage forms and in the treatment of neurodegeneration disorders, infections, dementias, inflammation, and injuries.

### BACKGROUND OF THE INVENTION

A co-crystal is a crystalline material comprising molecules of two or more different compounds in a neutral state and in a fixed stoichiometric ratio in a crystal lattice. The molecules of two or more different compounds are not covalently bound to each other and do not interact ionically in the co-crystal. Rather, the molecules of two or more different compounds interact nonionically. The co-crystal is solid at 25° C.

A co-crystal has physicochemical properties that are different from those of the compounds in its crystal lattice. For example, molecular weight, surface area, polarizability, van der Waals volume, melting point, solubility, bioavailability, hygroscopicity, stability, and/or permeability of the co-crystal may be different from the melting point, solubility, bioavailability, hygroscopicity, stability, permeability of the compounds in the crystal lattice of the co-crystal.

Without doing various empirical studies (e.g., solubility, melting points, and stability, etc.), it is currently impossible to predict which combination of compounds will crystallize as a co-crystal and, if it does, the resulting co-crystal structure.

Polymorphism is the ability of a substance to exist in multiple crystalline forms. Polymorphs exhibit different and distinct crystal structures and different and distinct physical and chemical properties, e.g., distinct X-ray powder diffraction patterns (XRPDs), solubilities, melting points, thermal behaviors, and stabilities.

It is currently impossible to predict whether a particular co-crystal has polymorphs and, if it does, physicochemical properties of the polymorphs and which polymorphs will be thermodynamically stable.

As a result, the discovery and selection of an appropriate polymorph of a co-crystal form to satisfy particular physicochemical property requirements is a non-trivial process.

### SUMMARY OF THE INVENTION

It is an object of the invention to provide a polymorph of a co-crystal of itanapraced and nicotinamide.

It is an additional object of the invention to provide a method of preparing a polymorph of a co-crystal of itanapraced and nicotinamide.

It is a further object of the invention to provide a pharmaceutical composition comprising a polymorph of a co-crystal of itanapraced and nicotinamide.

It is a further object of the invention to provide a dosage form comprising a polymorph of a co-crystal of itanapraced and nicotinamide.

It is another object of the present invention to provide a method for preventing, inhibiting, reducing, eliminating, protecting or delaying the onset of acute and chronic neurodegeneration disorders, including, e.g., mild cognitive impairment, dementias, neurologic injury, and neurologic inflammation, in a subject in need thereof.

It is another object of the present invention to overcome or ameliorate at least one of the disadvantages of the prior art, or to provide a useful alternative.

In accordance with the above objects and others, the invention is directed to a polymorph of a co-crystal of itanapraced and nicotinamide that could be produced at a gram scale and is stable enough to be used in pharmaceutical compositions and pharmaceutical dosage forms and be stable enough to be used as a seed crystal to synthesize the polymorph in bulk.

The polymorph comprises a crystal lattice comprising itanapraced and nicotinamide in a stoichiometric ratio of about 1:1, wherein itanapraced and nicotinamide are not covalently or ionically bound to each other in the crystal lattice, and are associated with each other only by non-ionic and noncovalent interactions. The chemical structure of the polymorph is:

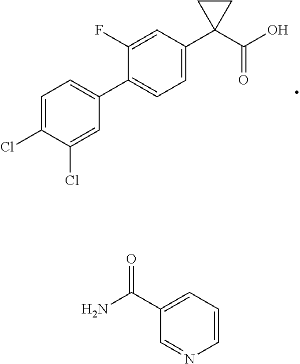 [SMILES: O=C(O)C1(c2ccc(-c3ccc(Cl)c(Cl)c3)c(F)c2)CC1 ; NC(=O)c1cccnc1]

The X-ray Powder Diffraction Pattern (XRPD) of the polymorph comprises peaks at 17.8°, 5.7°, 18.2°, 17.1°, 37.0°, and 28.6°, expressed in 2θ produced from a Cu radiation source (λ=1.54 Å after Ni filtering). The XRPD of the polymorph may comprise one or more additional peaks at positions selected from the group consisting of 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, and 14.2°, expressed in 2θ produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, and 31.5°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, 31.5°, and 22.8°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, 31.5°, 22.8°, and 13.9°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, 31.5°, 22.8°, 13.9°, and 25.7°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, 31.5°, 22.8°, 13.9°, 25.7°, and 35.9°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, 31.5°, 22.8°, 13.9°, 25.7°, and 35.9°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, 31.5°, 22.8°, 13.9°, 25.7°, 35.9°, and 22.3°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, 31.5°, 22.8°, 13.9°, 25.7°, 35.9°, 22.3°, and 19.7°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, 31.5°, 22.8°, 13.9°, 25.7°, 35.9°, 22.3°, 19.7°, and 29.0°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, and 37.4°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, and 39.8°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, and 15.1°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, and 29.5°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, and 20.1°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, and 11.4°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, and 24.1°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, and 8.5°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, and 16.5°, expressed in 2θ, produced from a Cu radiation source (λ=1.54 Å after Ni filtering).

In certain embodiments, the X-ray Powder Diffraction Pattern (XRPD) of the polymorph comprises peaks at 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering).

In certain embodiments, the polymorph has an X-ray Powder Diffraction Pattern (XRPD) that is substantially the same as the XRPD shown in FIG. 8.

The present invention is also directed to a crystalline system comprising itanapraced and nicotinamide in a stoichiometric ratio of about 1:1, wherein itanapraced and nicotinamide are linked by hydrogen bonding and other non-covalent and non-ionic interactions, the crystalline system is solid at 25° C. and has an X-ray Powder Diffraction Pattern (XRPD) having at least five peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering).

The present invention is also directed to a crystalline system comprising itanapraced and nicotinamide in a stoichiometric ratio of about 1:1, wherein itanapraced and nicotinamide are linked by hydrogen bonding and other non-covalent and non-ionic interactions, the crystalline system is solid at 25° C. and has an X-ray Powder Diffraction Pattern (XRPD) having at least six peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering).

The present invention is also directed to a crystalline system comprising itanapraced and nicotinamide in a stoichiometric ratio of about 1:1, wherein itanapraced and nicotinamide are linked by hydrogen bonding and other non-covalent and non-ionic interactions, the crystalline system is solid at 25° C. and has an X-ray Powder Diffraction Pattern (XRPD) having at least seven peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering).

The present invention is also directed to a crystalline system comprising itanapraced and nicotinamide in a stoichiometric ratio of about 1:1, wherein itanapraced and nicotinamide are linked by hydrogen bonding and other non-covalent and non-ionic interactions, the crystalline system is solid at 25° C. and has an X-ray Powder Diffraction Pattern (XRPD) having at least eight peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering).

The present invention is also directed to a crystalline system comprising itanapraced and nicotinamide in a stoichiometric ratio of about 1:1, wherein itanapraced and nicotinamide are linked by hydrogen bonding and other non-covalent and non-ionic interactions, the crystalline system is solid at 25° C. and has an X-ray Powder Diffraction Pattern (XRPD) having at least nine peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering).

The present invention is also directed to a crystalline system comprising itanapraced and nicotinamide in a stoichiometric ratio of about 1:1, wherein itanapraced and nicotinamide are linked by hydrogen bonding and other non-covalent and non-ionic interactions, the crystalline system is solid at 25° C. and has an X-ray Powder Diffraction Pattern (XRPD) having at least ten peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering).

The present invention is also directed to a crystalline system containing itanapraced and nicotinamide in a stoichiometric ratio of about 1:1, wherein itanapraced and nicotinamide are linked by hydrogen bonding and other non-covalent and non-ionic interactions, the crystalline system is solid at 25° C. and has an X-ray Powder Diffraction Pattern (XRPD) that is substantially the same as the XRPD shown in FIG. 8.

A crystalline system in accordance with the invention may consist essentially of CSPNCT2 and be polymorphically pure or it may comprise CSPNCT2 in combination with other polymorphs of a co-crystal of itanapraced and nicotinamide (e.g., a mixture of CSPNCT2 and CSPNCT1). The CSPNCT1 may comprise at least five peaks at positions selected from the group consisting of 20.0°, 18.5°, 18.2°, 14.6°, 16.7°, 14.9°, 22.0°, 24.2°, 26.1°, 30.0°, 26.1°, 20.3°, 34.0°, 22.3°, 15.6°, 26.5°, 24.5°, 30.6°, 28.9°, 27.8°, 29.4°, 36.8°, 36.5°, 19.0°, 32.4°, 7.5°, 31.6°, 39.1°, 13.9°, 21.6°, 19.4°, 11.1°, 25.1°, 23.5°, 36.0°, 35.7°, and 35.2°, expressed in 2θ, produced from a Cu radiation source (λ=1.54 Å after Ni filtering). The CSPNCT1 may comprise at least six peaks at positions selected from the group consisting of 20.0°, 18.5°, 18.2°, 14.6°, 16.7°, 14.9°, 22.0°, 24.2°, 26.1°, 30.0°, 26.1°, 20.3°, 34.0°, 22.3°, 15.6°, 26.5°, 24.5°, 30.6°, 28.9°, 27.8°, 29.4°, 36.8°, 36.5°, 19.0°, 32.4°, 7.5°, 31.6°, 39.1°, 13.9°, 21.6°, 19.4°, 11.1°, 25.1°, 23.5°, 36.0°, 35.7°, and 35.2°, expressed in 2θ, produced from a Cu radiation source (λ=1.54 Å after Ni filtering). The CSPNCT1 may comprise at least seven peaks at positions selected from the group consisting of 20.0°, 18.5°, 18.2°, 14.6°, 16.7°, 14.9°, 22.0°, 24.2°, 26.1°, 30.0°, 26.1°, 20.3°, 34.0°, 22.3°, 15.6°, 26.5°, 24.5°, 30.6°, 28.9°, 27.8°, 29.4°, 36.8°, 36.5°, 19.0°, 32.4°, 7.5°, 31.6°, 39.1°, 13.9°, 21.6°, 19.4°, 11.1°, 25.1°, 23.5°, 36.0°, 35.7°, and 35.2°, expressed in 2θ, produced from a Cu radiation source (λ=1.54 Å after Ni filtering). The CSPNCT1 may comprise at least eight peaks at positions selected from the group consisting of 20.0°, 18.5°, 18.2°, 14.6°, 16.7°, 14.9°, 22.0°, 24.2°, 26.1°, 30.0°, 26.1°, 20.3°, 34.0°, 22.3°, 15.6°, 26.5°, 24.5°, 30.6°, 28.9°, 27.8°, 29.4°, 36.8°, 36.5°, 19.0°, 32.4°, 7.5°, 31.6°, 39.1°, 13.9°, 21.6°, 19.4°, 11.1°, 25.1°, 23.5°, 36.0°, 35.7°, and 35.2°, expressed in 2θ, produced from a Cu radiation source (λ=1.54 Å after Ni filtering). The CSPNCT1 may comprise at least nine peaks at positions selected from the group consisting of 20.0°, 18.5°, 18.2°, 14.6°, 16.7°, 14.9°, 22.0°, 24.2°, 26.1°, 30.0°, 26.1°, 20.3°, 34.0°, 22.3°, 15.6°, 26.5°, 24.5°, 30.6°, 28.9°, 27.8°, 29.4°, 36.8°, 36.5°, 19.0°, 32.4°, 7.5°, 31.6°, 39.1°, 13.9°, 21.6°, 19.4°, 11.1°, 25.1°, 23.5°, 36.0°, 35.7°, and 35.2°, expressed in 2θ, produced from a Cu radiation source (λ=1.54 Å after Ni filtering). The CSPNCT1 may comprise at least ten peaks at positions selected from the group consisting of 20.0°, 18.5°, 18.2°, 14.6°, 16.7°, 14.9°, 22.0°, 24.2°, 26.1°, 30.0°, 26.1°, 20.3°, 34.0°, 22.3°, 15.6°, 26.5°, 24.5°, 30.6°, 28.9°, 27.8°, 29.4°, 36.8°, 36.5°, 19.0°, 32.4°, 7.5°, 31.6°, 39.1°, 13.9°, 21.6°, 19.4°, 11.1°, 25.1°, 23.5°, 36.0°, 35.7°, and 35.2°, expressed in 2θ, produced from a Cu radiation source (λ=1.54 Å after Ni filtering).

The present invention is also directed in part to a method of synthesizing a polymorph of a co-crystal of itanapraced and nicotinamide, the method comprising dissolving itanapraced and nicotinamide in a solvent to form a solution and precipitating the polymorph from the solution, wherein the polymorph has an X-ray Powder Diffraction Pattern (XRPD) having at least five peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering). The solvent may, e.g., be aprotic polar, aromatic apolar or lightly polar, or electron pair donor. In certain em selected from a group comprising dichloromethane, ethyl acetate, acetone, acetonitrile, ethanol, methanol, methyl tert-butyl ether, 2-butanol, toluene, water, and mixtures of at least two of any of the foregoing.

The present invention is also directed in part to a method of synthesizing a polymorph of a co-crystal of itanapraced and nicotinamide, the method comprising dissolving itanapraced and nicotinamide in dichloromethane to form a solution and precipitating the polymorph of the co-crystal of itanapraced and nicotinamide from the solution. To facilitate the dissolution of itanapraced and nicotinamide, the solution may, e.g., be heated from room temperature (25° C.) to about 50° C., and then cooled to a temperature between about 15° C. and about 40° C. to precipitate the polymorph of a co-crystal of itanapraced and nicotinamide. In some of the embodiments, the solution is held at 20-25° C. for no less than 1 hour. In some of the embodiments the precipitated polymorph is dried for at least 24 hours at full vacuum and 20-25° C.

The present invention is also directed in part to a method of synthesizing a polymorph of a co-crystal of itanapraced and nicotinamide, the method comprising dissolving itanapraced and nicotinamide in dichloromethane to form a solution and evaporating dichloromethane, thereby providing the polymorph.

The present invention is also directed in part to a method of synthesizing a polymorph of a co-crystal of itanapraced and nicotinamide, the method comprising dissolving itanapraced and nicotinamide in dichloromethane to form a solution and spray drying or lyophilizing the solution, thereby providing the polymorph.

The present invention is further directed in part to a method of synthesizing a polymorph of a co-crystal of itanapraced and nicotinamide, the method comprising dissolving itanapraced and nicotinamide in dichloromethane or ethyl acetate to form a solution, seeding the solution with a seed crystal of the polymorph, and precipitating the polymorph of the co-crystal of itanapraced and nicotinamide from the solution.

The polymorph of a co-crystal of itanapraced and nicotinamide synthesized in the methods of the invention has an X-ray Powder Diffraction Pattern (XRPD) having at least five peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering). In some of the embodiments of the invention, the polymorph having the X-ray Powder Diffraction Pattern (XRPD) comprising the three peaks is the only polymorph synthesized by the methods of invention.

The polymorph synthesized in the methods of the invention is stable enough to be used as an API in a pharmaceutical composition or a pharmaceutical dosage form or as a seed crystal in a solvent to manufacture the polymorph on a gram scale (e.g., in bulk). For example, the polymorph synthesized in the methods of the invention may also be used as a seed crystal in dichloromethane or another solvent (e.g., ethyl acetate) to manufacture the polymorph on a gram scale or in bulk.

In certain embodiments of the invention, the polymorph synthesized in the methods of the invention is synthesized in bulk, e.g., in amount from about from about 1 kg to 1000 kg, 10 kg to about 1000 kg or from about 10 kg to about 500 kg.

The present invention is further directed to a pharmaceutical composition consisting essentially of a polymorph of a co-crystal of itanapraced and nicotinamide, the polymorph having an X-ray Powder Diffraction Pattern (XRPD) having at least five peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering). In one embodiment, the polymorph has an X-ray Powder Diffraction Pattern (XRPD) that is substantially the same as the XRPD shown in FIG. 8.

The present invention is also directed to a pharmaceutical composition comprising a mixture of polymorphs of a co-crystal of itanapraced and nicotinamide, wherein one of the polymorphs has an X-ray Powder Diffraction Pattern (XRPD) having at least five peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering).

The present invention is further directed to a pharmaceutical composition comprising a mixture of polymorphs of a co-crystal of itanapraced and nicotinamide, wherein one of the polymorphs has an X-ray Powder Diffraction Pattern (XRPD) that is substantially the same as the XRPD shown in FIG. 8.

In addition, the present invention is also directed to a pharmaceutical dosage form comprising (i) a polymorph of a co-crystal of itanapraced and nicotinamide and (ii) one or more pharmaceutically acceptable excipient(s), wherein the polymorph has an X-ray Powder Diffraction Pattern (XRPD) having at least five peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering). The polymorph of a co-crystal of itanapraced and nicotinamide may, for example, comprise from about 1% to about 99% of the pharmaceutical dosage form by weight, with the one or more pharmaceutically acceptable excipient(s) comprising the remainder of the pharmaceutical dosage form by weight.

The present invention is further directed to a pharmaceutical dosage form comprising (i) a polymorph of a co-crystal of itanapraced and nicotinamide and (ii) one or more pharmaceutically acceptable excipient(s), wherein the polymorph has an X-ray Powder Diffraction Pattern (XRPD) that is substantially the same as the XRPD shown in FIG. 8.

The present invention is also directed to a pharmaceutical dosage form comprising (i) a mixture of polymorphs of a co-crystal of itanapraced and nicotinamide, wherein one of the polymorphs has an X-ray Powder Diffraction Pattern (XRPD) having at least five peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering), and (ii) one or more pharmaceutically acceptable excipients.

The present invention is also directed to a pharmaceutical dosage form comprising (i) a mixture of polymorphs of a co-crystal of itanapraced and nicotinamide, wherein one of the polymorphs has an X-ray Powder Diffraction Pattern (XRPD) having at least six peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering), and (ii) one or more pharmaceutically acceptable excipients.

The present invention is also directed to a pharmaceutical dosage form comprising (i) a mixture of polymorphs of a co-crystal of itanapraced and nicotinamide, wherein one of the polymorphs has an X-ray Powder Diffraction Pattern (XRPD) having at least seven peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering), and (ii) one or more pharmaceutically acceptable excipients.

The present invention is also directed to a pharmaceutical dosage form comprising (i) a mixture of polymorphs of a co-crystal of itanapraced and nicotinamide, wherein one of the polymorphs has an X-ray Powder Diffraction Pattern (XRPD) having at least eight peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering), and (ii) one or more pharmaceutically acceptable excipients.

The present invention is also directed to a pharmaceutical dosage form comprising (i) a mixture of polymorphs of a co-crystal of itanapraced and nicotinamide, wherein one of the polymorphs has an X-ray Powder Diffraction Pattern (XRPD) having at least nine peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering), and (ii) one or more pharmaceutically acceptable excipients.

The present invention is also directed to a pharmaceutical dosage form comprising (i) a mixture of polymorphs of a co-crystal of itanapraced and nicotinamide, wherein one of the polymorphs has an X-ray Powder Diffraction Pattern (XRPD) having at least ten peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering), and (ii) one or more pharmaceutically acceptable excipients.

The present invention is also directed to a pharmaceutical dosage form comprising (i) a mixture of polymorphs of a co-crystal of itanapraced and nicotinamide, wherein one of the polymorphs has an X-ray Powder Diffraction Pattern (XRPD) that is substantially the same as the XRPD shown in FIG. 8, and (ii) one or more pharmaceutically acceptable excipients.

The present invention in further directed in part to a method for preventing, inhibiting, reducing, eliminating, protecting or delaying the onset of acute and chronic neurodegeneration disorders, including, e.g., mild cognitive impairment, dementias, neurologic injury, and neurologic inflammation, in a subject in need thereof comprising administering to a subject in need thereof a therapeutically effective amount of a polymorph of a co-crystal of itanapraced and nicotinamide, the polymorph having an X-ray Powder Diffraction Pattern (XRPD) having at least five peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering).

The present invention in further directed in part to a method for preventing, inhibiting, reducing, eliminating, protecting or delaying the onset of acute and chronic neurodegeneration disorders, including, e.g., mild cognitive impairment, dementias, neurologic injury, and neurologic inflammation, in a subject in need thereof comprising administering to a subject in need thereof a therapeutically effective amount of a polymorph of a co-crystal of itanapraced and nicotinamide, the polymorph having an X-ray Powder Diffraction Pattern (XRPD) that is substantially the same as the XRPD shown in FIG. 8.

In the method for preventing, inhibiting, reducing, eliminating, protecting or delaying the onset of acute and chronic neurodegeneration disorders, including, e.g., mild cognitive impairment, dementias, neurologic injury, and neurologic inflammation, the polymorph may be combined with one or more pharmaceutically acceptable excipient(s) disclosed in the present specification to form a pharmaceutical composition or a pharmaceutical dosage form, prior to the administration.

A method for preventing, inhibiting, reducing, eliminating, protecting or delaying the onset of acute and chronic neurodegeneration disorders, including, e.g., mild cognitive impairment, dementias, neurologic injury, and neurologic inflammation, in accordance with the invention comprises administering a pharmaceutically acceptable dosage form comprising a therapeutically effective amount of a polymorph of a co-crystal of itanapraced and nicotinamide, the polymorph having an X-ray Powder Diffraction Pattern (XRPD) having at least five peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering).

A method for preventing, inhibiting, reducing, eliminating, protecting or delaying the onset of acute and chronic neurodegeneration disorders, including, e.g., mild cognitive impairment, dementias, neurologic injury, and neurologic inflammation, in accordance with the invention comprises administering a pharmaceutically acceptable dosage form comprising a therapeutically effective amount of a polymorph of a co-crystal of itanapraced and nicotinamide, the polymorph having an X-ray Powder Diffraction Pattern (XRPD) that is substantially the same as the XRPD shown in FIG. 8.

### Definitions

As used herein, in the context of the present disclosure, each of the following terms has the meaning associated with it in this section.

The articles “a” and “an” refer to one or to more than one (i.e. to at least one) of the grammatical object of the article. By way of example, “an element” means one element or more than one element.

The term “about” means a value within 20% (±20%) of the value recited immediately after the term “about,” including the value equal to the upper limit (i.e., +20%) and the value equal to the lower limit (i.e., −20%) of this range. For example, the phrase “about 100” encompasses any numeric value that is between 80 and 120, including 80 and 120.

The term “bulk” means an amount of material of at least 500 mg. In certain embodiments, the amount can be from about 1 kg to 1000 kg, 10 kg to about 1000 kg or from about 10 kg to about 500 kg.

The term “co-crystal” means a crystalline material composed of molecules of two or more different compounds in a crystal lattice, one or more of which is the API(s), in a defined stoichiometric ratio within the same crystal lattice that are associated by nonionic and noncovalent bonds.

The term “coformer” means a component that interacts nonionically with the API in the crystal lattice that is typically nonvolatile and is not a solvent.

“Effective amount” or “therapeutically effective amount” are used interchangeably herein, and refer to an amount of a compound, formulation, material, or composition, as described herein effective to achieve a particular biological result. Such results may include, but are not limited to, the treatment of a disease or condition as determined by any means suitable in the art.

The term “polymorphs” means different crystalline forms of co-crystals of itanapraced and nicotinamide.

The expression “polymorphically pure” or “substantially free of any other solid state forms” means that the solid state form contains about 20% (w/w) or less, about 10% (w/w) or less, about 5% (w/w) or less, about 2% (w/w) or less, about 1% (w/w) or less, or about 0% of any other forms of the subject compound, as measured by XRPD.

The expression “substantially pure crystalline form” means a crystalline form characterized by XRPD that contains no more than traces of the signals relating to other crystalline forms. Preferably, the presence of such signals is equal to or below the limit of detection (LOD) of the method used and therefore, in the majority of the cases described herein, the expression “substantially pure crystalline form” means a crystalline form with a purity of at least 90%. The term “main peaks” used herein means peaks with a relative intensity≥ 1.7%.

The term “pharmaceutical composition” refers to a polymorph of a co-crystal of itanapraced and nicotinamide or a mixture of the polymorh with other chemical components, such as carriers, stabilizers, diluents, dispersing agents, suspending agents, thickening agents, and/or excipients, prior to the polymorph or the mixture being incorporated into a pharmaceutical dosage form.

The term “pharmaceutical dosage form” refers to a product comprising the polymorph of the invention as will be administered to a subject in need thereof (e.g., a tablet). A pharmaceutical dosage form contains a fixed amount of the polymorph of the invention per dosage form.

“Pharmaceutically acceptable” refers to those properties and/or substances that are acceptable to a subject (e.g., a human) from a pharmacological/toxicological point of view and to the manufacturing pharmaceutical chemist from a physical/chemical point of view regarding composition, formulation; stability, subject's acceptance and bioavailability.

The term “treat” or “treating” includes but is not limited to, alleviation or amelioration of one or more symptoms or conditions; diminishment of extent of disease, disorder, or condition; stabilized (i.e., not worsening) state of disease, disorder, or condition; preventing spread of disease, disorder, or condition (e.g., delay or slowing the progress of the disease, disorder, or condition; amelioration or palliation of the disease, disorder, or condition; and remission (whether partial or total), whether detectable or undetectable.

“Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

As used herein, “preventing” includes preventing the initiation of a disease and/or reducing the severity or intensity of the disease.

As used herein, “alleviate” is used interchangeably with the term “treat.” Treating a disease, disorder or condition may or may not include complete eradication or elimination of the symptom.

The term “salt” as used herein means a compound formed by the ionizable reaction between an acid and a base, where one component is an active pharmaceutical ingredient (API).

The term “therapeutic” as used herein means a treatment and/or prophylaxis.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “substantially the same as” with respect to the X-ray Powder Diffraction Pattern is intended to indicate that the 2-theta angle values of the X-ray powder diffraction patterns may vary slightly) (±2°) and relative peak intensities from sample preparation to sample preparation and from one machine to another, from one sample to another, or as a result of slight variations in sample preparation and measurement conditions utilized. The X-ray Powder Diffraction Patterns shown in FIGS. 8 and 9 are substantially the same. In contrast, the X-ray Powder Diffraction Pattern shown in FIG. 7 is not substantially the same the X-ray Powder Diffraction Pattern shown in FIGS. 8 and 9. The peak positions shown in the X-ray Powder Diffraction Pattern and described in the accompanying description are not to be construed as absolute values.

The abbreviation “CSP-1103” means itanapraced.

The abbreviation “NCT” means nicotinamide.

The abbreviation “CSPNCT” means a co-crystal of itanapraced and nicotinamide

The abbreviation “CSPNCT1” means a polymorph of a co-crystal of itanapraced and nicotinamide having an X-ray Powder Diffraction Pattern (XRPD) that is substantially the same as the XRPD shown in FIG. 7.

The abbreviation “CSPNCT2” means a polymorph of a co-crystal of itanapraced and nicotinamide having an XRPD that is substantially the same as the XRPD shown in FIGS. 8 and 9. While there are differences in relative peak intensities in FIGS. 8 and 9, the main peak positions between the peak positions in FIGS. 8 and 9 are not changed, which indicates that the same solid form (i.e., the same polymorph) is being analyzed in FIGS. 8 and 9.

In the context of the present disclosure, CSPNCT1 and CSPNCT2 are different and distinct polymorphs of a co-crystal of itanapraced and nicotinamide. CSPNCT1 and CSPNCT2 have X-ray Powder Diffraction Patterns that are different and distinct.

The abbreviation “DCM” means dichloromethane.

The abbreviation “EtOAc” means ethyl acetate.

The abbreviation “DMSO” means dimethyl sulfoxide.

The abbreviation “MeOH” means methanol.

The abbreviation “2-Me THF” means 2-methyl tetrahydrofuran.

The abbreviation “ACN” means acetonitrile.

The abbreviation “EtOH” means ethanol.

The abbreviation “IPA” means isopropanol, or 2-propanol.

The abbreviation “NMR” means Nuclear Magnetic Resonance (1H NMR: Proton NMR).

The abbreviation “XRPD” means X-Ray Powder Diffraction.

For the purposes of the present invention, the term “non-hygroscopic” means that the molecule gains or loses less than 0.20% w/w, at 25° C. between 5-95% RH (relative humidity).

For the purposes of the present invention, the term “slightly hygroscopic” means that the molecule gains or loses more than 0.20% w/w but less than 2% w/w- at 25° C. between 5-95% RH (relative humidity).

In the context of the present disclosure, the “neurodegenerative condition” includes Parkinson's disease (PD), Alzheimer's disease (AD), Multiple Sclerosis (MS), juvenile neuronal ceroid lipofuscinosis (JNCL) (Batten disease type-3), age-related macular degeneration (AMD); dementias (e.g., MCI), neurological infection, neurologic injury (Traumatic Brain Injury (TBI)) and neurologic inflammation. The “neurodegenerative condition” also includes tauopathies, especially Amyotrophic Lateral Sclerosis (ALS), Pick's disease, Frontal Temporal Dementia (FTD) and Progressive Supranuclear Palsy (PSP), as well as brain hypoxia.

In the context of the present invention, “AUC” is the area under the plot of plasma concentration of itanapraced (not logarithm of the concentration) against time after itanapraced administration. The area is conveniently determined by the “trapezoidal rule”: the data points are connected by straight line segments, perpendiculars are erected from the abscissa to each data point, and the sum of the areas of the triangles and trapezoids so constructed is computed. When the last measured concentration (Cₙ, at time tₙ) is not zero, the AUC from tₙ to infinite time is estimated by Cₙ/kₑₗ.

The AUC is of particular use in estimating bioavailability of itanapraced, and in estimating total clearance of itanapraced (CIT). Following single intravenous doses, AUC-D/C1T, for single compartment systems obeying first-order elimination kinetics; alternatively, AUC=C₀/kₑₗ. With routes other than the intravenous, for such systems, AUC=F·D/C1T, where F is the availability of the itanapraced.

In the context of the present disclosure, “dose response” is the quantitative relationship between the magnitude of response and the dose inducing the response and may be measured by conventional means known in the art. The curve relating effect (as the dependent variable) to dose (as the independent variable) for an itanapraced-cell system is the “dose-response curve”. Typically, the dose-response curve is the measured response to an itanapraced plotted against the dose of the itanapraced (mg/kg) given. The dose response curve can also be a curve of AUC against the dose of the itanapraced given.

### BRIEF DESCRIPTION OF THE DRAWINGS

FIG. 1 shows XRPD Patterns of Initial Co-Crystal Probe.

FIG. 2 shows XRPD Patterns of Crystallization Pilots (Seeding).

FIG. 3 shows XRPD Patterns of CSPNCT2 Scale Up.

FIG. 4 shows XRPD Zoom of CSPNCT2 (EtOAc) and CSPNCT2 (DCM).

FIG. 5 shows 1H NMR (Aromatic Zoom) of CSPNCT2 isolated from DCM.

FIG. 6 shows 1H NMR (Aromatic Zoom) of CSPNCT2 isolated from Ethyl Acetate.

FIG. 7 shows XRPD Pattern of CSPNCT1.

FIG. 8 shows XRPD Pattern of CSPNCT2 (DCM).

FIG. 9 shows XRPD Pattern of CSPNCT2 (EtOAc).

FIG. 10 shows an overlay of the XRPD Pattern of CSPNCT2 (DCM) and XRPD Pattern of CSPNCT2 (EtOAc), which have been normalized at maximum peak intensity. While there are differences in relative peak intensity, the main peak positions between the peak positions of the XRPD Pattern of CSPNCT2 (DCM) and XRPD Pattern of CSPNCT2 (EtOAc) are not changed. This confirms that CSPNCT2 (DCM) and CSPNCT2 (EtOAc) is the same solid form (i.e., the same polymorph). The differences in relative peak intensity could be the result of different levels of grinding between the samples prior to mounting for X-ray analysis and the way samples were laid on the plate.

### DETAILED DESCRIPTION

Polymorphs and crystalline systems in accordance with the invention are new solid forms of co-crystals of itanapraced and nicotinamide.

A polymorph of a co-crystal of itanapraced and nicotinamide of the invention comprises a crystal lattice comprising itanapraced and nicotinamide in a stoichiometric ratio of about 1:1, wherein itanapraced and nicotinamide are not covalently or ionically bound to each other in the crystal lattice, and are associated with each other only by non-ionic and noncovalent interactions.

The chemical structure of the polymorph is:

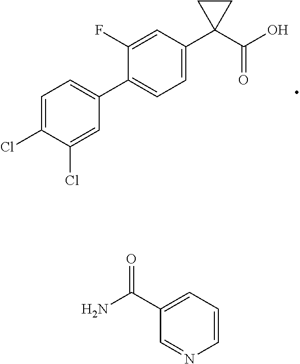 [SMILES: O=C(O)C1(c2ccc(-c3ccc(Cl)c(Cl)c3)c(F)c2)CC1 ; NC(=O)c1cccnc1]

The X-ray Powder Diffraction Pattern (XRPD) of the polymorph has at least five peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering).

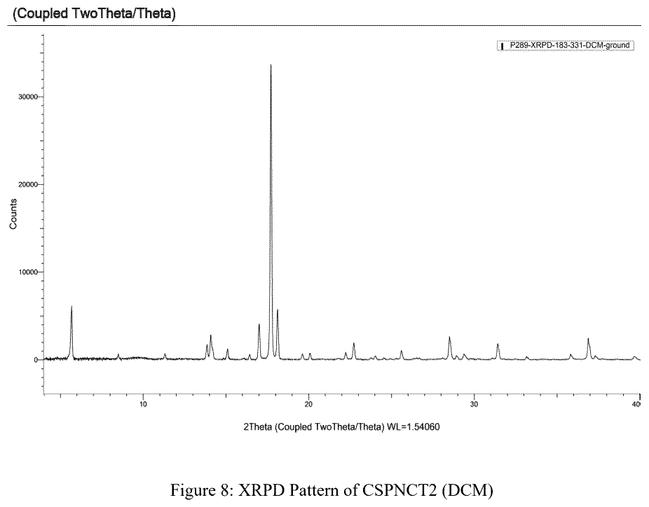

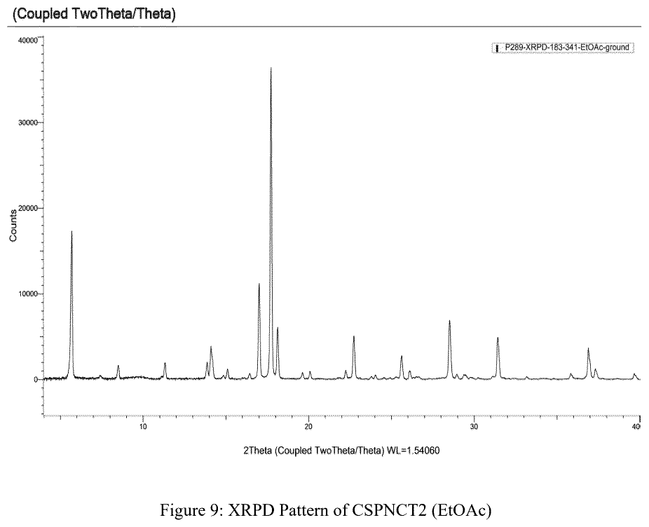

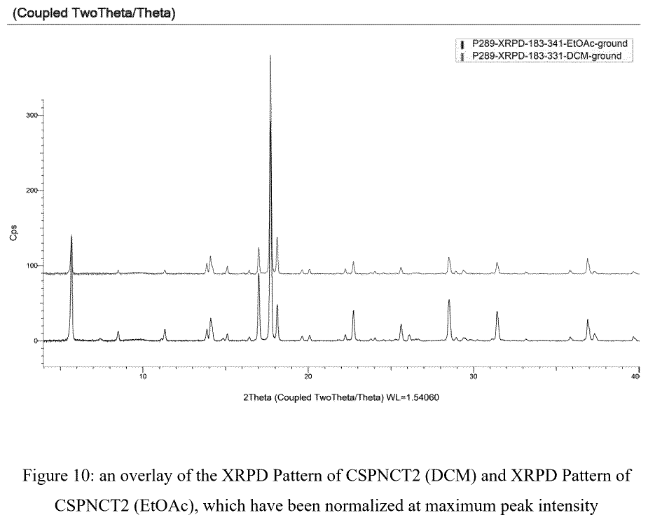

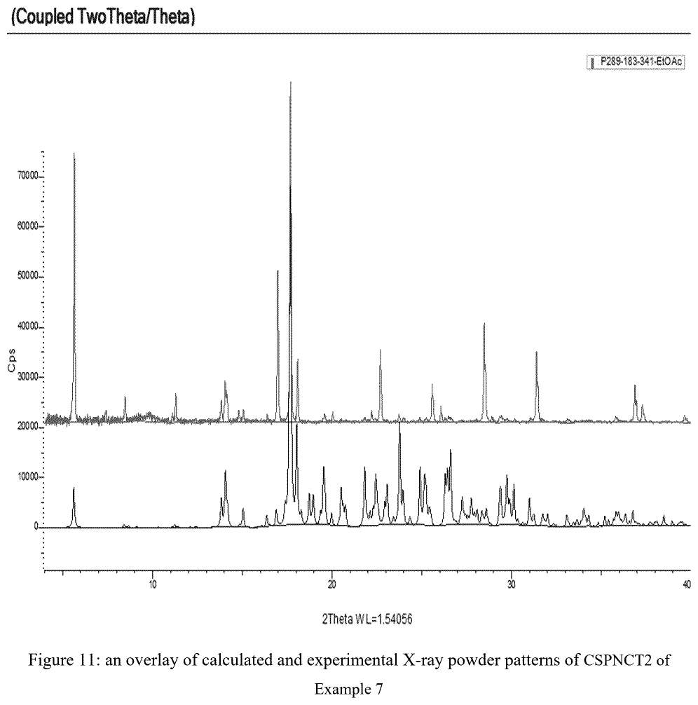

In one embodiment, the polymorph has an X-ray Powder Diffraction Pattern (XRPD) that is substantially the same as the XRPD shown in FIG. 8.

A crystalline system in accordance with the invention comprises itanapraced and nicotinamide in a stoichiometric ratio of about 1:1, wherein itanapraced and nicotinamide are linked by hydrogen bonding and other non-covalent and non-ionic interactions, the crystalline system is solid at 25° C. and has an X-ray Powder Diffraction Pattern (XRPD) having at least five peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering). In one embodiment, the crystalline system has an XPRD that is substantially the same as the XRPD shown in FIG. 8. A crystalline system in accordance with the invention may consist essentially of CSPNCT2 and be polymorphically pure or it may comprise CSPNCT2 in combination with other polymorphs of a co-crystal of itanapraced and nicotinamide (e.g., a mixture of CSPNCT2 and CSPNCT1).

### Itanapraced

Itanapraced, 1-(3′,4′-dichloro-2-fluoro[1,1′-biphenyl]-4-yl)-cyclopropanecarboxylic acid) (aka CSP-1103, formerly CHF 5074), is a small molecule with good oral bioavailability, a long plasma half-life and substantial penetration into the brain.

Itanapraced has the following chemical structure:

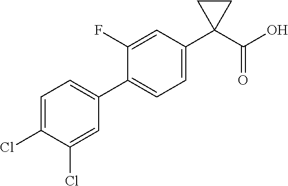 [SMILES: O=C(O)C1(c2ccc(-c3ccc(Cl)c(Cl)c3)c(F)c2)CC1]

Molecular weight of itanapraced is 325.16.

Itanapraced belongs to a new class of drug compounds (“praceds”) that bind the amyloid precursor protein (APP) and inhibit the transcriptional activity of its intracellular domain (AICD).

In the cytoplasm of mammalian cells, AICD physically interacts with the transcription factor forkhead box O) (FoxO), which is a crucial downstream mediator of APP-induced cell death and locomotion defect; and also translocates with FoxO into the nucleus upon oxidative stress.

Under conditions of acute oxidative stress, AICD transcriptional activity may cause cell damage by interacting with FOXO3a, a critical component of the physiological response mechanism to oxidative stress.

APP may therefore modulate FoxO-mediated cell death through AICD, which acts as a transcriptional co-activator of FoxO.

In addition, in neurons, astrocytes and microglia, APP may have a proinflammatory function.

Itanapraced may bind the amyloid precursor protein (APP) and inhibit the transcriptional activity of its intracellular domain (AICD). Itanapraced may also modulate microglia. Itanapraced may also inhibit inflammation. In some of the embodiments, nicotinamide may augment therapeutic activity of itanapraced.

Itanapraced may also modulate microglia.

Thus, Itanapraced may have utility in treatment of a broad range of disease indications pointing to a common mechanism linked by oxidative stress and neuroinflammation.

For example, itanapraced has been reported to have numerous beneficial effects in transgenic Alzheimer's mouse models (Imbimbo, 2007, 2009; Lanzillotta 2011), in a rat traumatic brain injury model (Lin et al., 2017) and in mouse models of Parkinson's Disease (manuscript submitted) and Juvenile Batten disease (unpublished). In addition, itanapraced prevented the accumulation of hypertrophic microglia in the injured brain and attenuated both neurological and acute lung injury in rats after TBI (Li 2017).

Itanapraced has been tested in over 200 subjects in several Phase 1 studies and a Phase 2 study in mild cognitive impairment (MCI), with favorable results (Ross, 2013). In addition to good safety and tolerability, itanapraced produced beneficial dose-related CSF differences in two key neuroinflammatory mediators, TNF-α and soluble CD40L, as well as in levels of total tau, a recognized marker of neurodegeneration. Patients also exhibited stable cognition throughout the duration of the trial.

In a Phase 2 study in patients with mild cognitive impairment (MCI) treated up to two years (double blind for 3 months; open label thereafter), itanapraced was found to be well tolerated and produced dose-related statistically significant reductions, in the brain, of two key neuroinflammatory mediators, soluble CD40 ligand and TNF-α as well as, total tau, a recognized marker of neurodegeneration. In addition, patients exhibited stable cognitive function throughout the long duration of the trial.

Itanapraced may also have utility in treatment of Parkinson's disease, Juvenile Batten disease, mild cognitive impairment, and LRRK-mediated neurotoxicity.

In addition, itanapraced may have utility in preventing, inhibiting or treating CNS infection(s) and neurological damage in humans following peripheral COVID-19 infection may also mitigate lung damage and loss of function.

### Nicotinamide

Nicotinamide (NCT) is a form of vitamin B3.

Nicotinamide has the following structure:

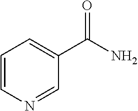

Molecular weight of nicotinamide is 122.13.

Nicotinamide is a precursor of (NAD) (+), which means cells can use a chemical reaction to turn nicotinamide into (NAD) (+). (NAD) (+) is a crucial component of the chemical reaction that mitochondria use to produce energy.

Nicotinamide is found in food and may, e.g., be used as a dietary supplement and medication. As a supplement, it may be used, e.g., by mouth to prevent and treat pellagra (niacin deficiency). While nicotinic acid (niacin) may also be used for this purpose, nicotinamide has the benefit of not causing skin flushing. As a cream, nicotinamide may be used, e.g., to treat acne.

Nicotinamide can be used a coformer to form a co-crystal with itanapraced.

In some embodiments, nicotinamide may be included in an amount sufficient to provide an improvement in hygroscopicity of itanapraced, as compared to hygroscopicity of the itanapraced without nicotinamide.

Depending on the embodiment and dose, nicotinamide may or may not have biological activity when polymorph of the invention is administered to a subject in need thereof.

### Utility of The Polymorphs of the Present Invention

The polymorphs of the present invention may comprise itanapraced and nicotinamide and may therefore be used for treatment of neurodegeneration disorders, infections, dementias, inflammation, and injuries.

As compared to itanapraced, the polymorph of the invention may provide a better bioavailability and/or better efficacy and/or improved stability.

### Dosage Forms

A pharmaceutical dosage form comprises an active pharmaceutical ingredient (API) and one or more pharmaceutically acceptable excipient(s). An API is an ingredient in a pharmaceutical dosage form that is biologically active (i.e., a drug). An “excipient” is an inert pharmaceutical ingredient that is used in a pharmaceutical formulation or a pharmaceutical dosage form.

Traditionally, a free base, a salt, a solvate, or a hydrate the ingredient was used as an API.

A salt is a chemical compound formed from the reaction of an acid with a base.

A solvate is a multicomponent crystalline solid molecular adduct formed by solvation (i.e., the combination of solvent molecules or ions with molecules of the solute). The solvate contains molecules or ions of the solute and molecules of solvent(s) in a crystal lattice structure.

A hydrate is a multicomponent crystalline solid molecular adduct formed by hydration (i.e., the combination of water molecules with molecules or ions of the solute). The hydrate contains molecules or ions of the solute and molecules of water incorporated in a crystal lattice structure.

Co-crystals are distinguishable from salts, because the components of co-crystals interact nonionically rather than ionically.

Co-crystals are distinguishable from solvates, because the second component of the co-crystal (a coformer) is not a solvent and typically is nonvolatile.

Co-crystals are distinguishable from hydrates, because the second component of the co-crystal (a coformer) is not water and typically is nonvolatile.

An API in the dosage forms of the present invention comprises a polymorph of a co-crystal of itanapraced and nicotinamide or a crystalline system comprising itanapraced and nicotinamide in a stoichiometric ratio of about 1:1, wherein itanapraced and nicotinamide are linked by hydrogen bonding and other non-covalent and non-ionic interactions, the crystalline system is solid at 25° C. The polymorph or the crystalline system has an XRPD having at least five peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering). The formulations and dosage forms of present invention may contain from about 3 mg to about 3500 mg of the polymorph or the crystalline system.

In the present invention, one or more polymorph(s) of a co-crystal of itanapraced and nicotinamide are formulated with one or more pharmaceutically acceptable excipient(s) into a pharmaceutically acceptable dosage form.

In one embodiment, one or more polymorph(s) of a co-crystal of itanapraced and nicotinamide is (are) formulated with one or more pharmaceutically acceptable excipient(s) into a pharmaceutically acceptable oral dosage form. Oral dosage forms may include but are not limited to, oral solid dosage forms and oral liquid dosage forms. Oral solid dosage forms may include but are not limited to, tablets, capsules, caplets, powders, pellets, multiparticulates, beads, spheres and/or any combinations thereof. These oral solid dosage forms may be formulated as immediate release, controlled release, sustained (extended) release or modified release formulations.

The oral solid dosage forms of the present invention may contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof. Each of these excipient(s) may, e.g., comprise from about 0.1% to about 99.9%, from about 0.5% to about 95%, from about 1% to about 95%, from about 2% to about 95%, from about 3% to about 95%, or from about 5% to about 95% of the formulation by weight.

In some embodiments, the solid dosage forms of the present invention may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations of the present invention may be administered as a single capsule or in multiple capsule dosage form. In some embodiments, the pharmaceutical formulation is administered in two, or three, or four, capsules or tablets.

The pharmaceutical solid dosage forms described herein can comprise the polymorph of the present invention as an API and one or more pharmaceutically acceptable excipient(s) such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the active agent(s) of the present invention formulation. In one embodiment, some or all of the active agent(s) of the present invention particles are coated. In another embodiment, some or all of the active agent(s) of the present invention particles are microencapsulated. In yet another embodiment, some or all of the active agent(s) of the present invention is amorphous material coated and/or microencapsulated with inert excipients. In still another embodiment, the active agent(s) of the present invention particles not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose (e.g., Avicel®, Avicel® PH101, Avicel® PH102, Avicel® PH105, etc.), cellulose powder, dextrose, dextrates, dextrose, dextran, starches, pregelatinized starch, hydroxypropylmethylcellulose (HPMC), hydroxypropylmethylcellulose phthalate, hydroxypropylmethylcellulose acetate stearate (HPMCAS), sucrose, xylitol, lactitol, mannitol, sorbitol, sodium chloride, polyethylene glycol, and the like.

If needed, suitable disintegrants for use in the solid dosage forms described herein include, but are not limited to, natural starch such as corn starch or potato starch, a pregelatinized starch such as National 1551 or Amijel®, or a sodium starch glycolate such as Promogel® or Explotab®, a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, Ac-Di-Sol, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder-filled capsule formulation, they aid in plug formation that can be filled into soft or hard shell capsules and in tablet formulation, binders ensure that the tablet remains intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like. In general, binder levels of 20-70% are used in powder-filled gelatin capsule formulations. Binder usage level in tablet formulations is a function of whether direct compression, wet granulation, roller compaction, or usage of other excipients such as fillers which itself can act as moderate binders are used. Formulators skilled in the art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumarate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Non-water-soluble diluents are compounds typically used in the formulation of pharmaceuticals, such as calcium phosphate, calcium sulfate, starches, modified starches and microcrystalline cellulose, and micro cellulose (e.g., having a density of about 0.45 g/cm3, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like. Wetting agents include surfactants.

Suitable surfactants for use in the solid dosage forms described herein include, for example, docusate and its pharmaceutically acceptable salts, sodium lauryl sulfate, sorbitan monooleate, polyoxyethylene sorbitan monooleate, polysorbates, poloxamers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 18000, vinylpyrrolidone/vinyl acetate copolymer (S630), sodium alginate, gums, such as, e.g., gum tragacanth and gum acacia, guar gum, xanthans, including xanthan gum, sugars, cellulosic, such as, e.g., sodium carboxymethylcellulose, methylcellulose, sodium carboxymethylcellulose, hydroxypropylmethylcellulose, hydroxyethylcellulose, polysorbate-80, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), butyl hydroxyanisole (BHA), sodium ascorbate, Vitamin E TPGS, ascorbic acid, sorbic acid and tocopherol.

Immediate-release formulations may be prepared by combining super disintegrant such as Croscarmellose sodium and different grades of microcrystalline cellulose in different ratios. To aid disintegration, sodium starch glycolate will be added.

In cases where the two (or more) drugs included in the fixed-dose combinations of the present invention are incompatible, cross-contamination can be avoided, e.g. by incorporation of the drugs in different drug layers in the oral dosage form with the inclusion of a barrier layer(s) between the different drug layers, wherein the barrier layer(s) comprise one or more inert/non-functional materials.

The above-listed excipients should be taken as merely examples and not limiting, of the types of excipients that can be included in solid dosage forms of the present invention. The amounts of such excipients can be readily determined by one skilled in the art, according to the particular properties desired.

Oral liquid dosage forms include, but are not limited to, solutions, emulsions, suspensions, and syrups. These oral liquid dosage forms may be formulated with any pharmaceutically acceptable excipient known to those of skill in the art for the preparation of liquid dosage forms. For example, water, glycerin, simple syrup, alcohol, and combinations thereof.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as but not limited to, an oil, water, an alcohol, and combinations of these pharmaceutically suitable surfactants, suspending agents, emulsifying agents, may be added for oral or parenteral administration. Suspensions may include oils. Such oils include, but are not limited to, peanut oil, sesame oil, cottonseed oil, corn oil, and olive oil. Suspensions may also contain esters of fatty acids such as ethyl oleate, isopropyl myristate, fatty acid glycerides, and acetylated fatty acid glycerides. Suspension formulations may include alcohols, such as, but not limited to, ethanol, isopropyl alcohol, hexadecyl alcohol, glycerol, and propylene glycol. Ethers, such as but not limited to, poly (ethylene glycol), petroleum hydrocarbons such as mineral oil and petrolatum; and water may also be used in suspension formulations.

In some embodiments, formulations are provided comprising the co-crystal of the present invention particles described herein and at least one dispersing agent or suspending agent for oral administration to a subject. The formulation may be a powder and/or granules for suspension, and upon admixture with water, a substantially uniform suspension is obtained. As described herein, the aqueous dispersion can comprise amorphous and non-amorphous the active agent(s) of the present invention particles of consisting of multiple effective particle sizes such that the active agent(s) of the present invention particles having a smaller effective particle size is absorbed more quickly and the active agent(s) of the present invention particles having a larger effective particle size are absorbed more slowly. In certain embodiments, the aqueous dispersion or suspension is an immediate-release formulation. In another embodiment, an aqueous dispersion comprising amorphous the active agent(s) of the present invention particles is formulated such that a portion of the active agent(s) of the present invention particles are absorbed within, e.g., about 3 hours after administration and about 90% of the active agent(s) of the present invention particles are absorbed within, e.g., about 10 hours after administration. In other embodiments, addition of a complexing agent to the aqueous dispersion results in a larger span of the active agent(s) of the present invention containing particles to extend the drug absorption phase such that 50-80% of the particles are absorbed in the first 3 hours and about 90% are absorbed by about 10 hours. Dosage forms for oral administration can be aqueous suspensions selected from the group including, but not limited to, pharmaceutically acceptable aqueous oral dispersions, emulsions, solutions, and syrups. See, e.g., Singh et al., Encyclopedia of Pharmaceutical Technology, 2nd Ed., pp. 754-757 (2002). In addition to the active agent(s) of the present invention particles, the liquid dosage forms may comprise excipients, such as (a) disintegrating agents; (b) dispersing agents; (c) wetting agents; (d) at least one preservative, (e) viscosity enhancing agents, (f) at least one sweetening agent, and (g) at least one flavoring agent.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropylcellulose ethers (e.g., HPC, HPC-SL, and HPC-L), hydroxypropylmethylcellulose and hydroxypropylmethylcellulose ethers (e.g. HPMC K100, HPMC K4M, HPMC K15M, and HPMC K100M), carboxymethylcellulose sodium, methylcellulose, hydroxyethylcellulose, hydroxypropylmethylcellulose phthalate, hydroxypropylmethylcellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween® 60 or 80; PEG; polyvinylpyrrolidone (PVP); hydroxypropyl cellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethylcellulose phthalate; hydroxypropylmethylcellulose acetate stearate; non-crystalline cellulose; magnesium aluminum silicate; triethanolamine; polyvinyl alcohol (PVA); 4-(1,1,3,3-tetramethyl butyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents (including surfactants) suitable for the aqueous suspensions and dispersions described herein are known in the art and include, but are not limited to, acetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carpool 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphatidylcholine and the like.

Suitable preservatives for the aqueous suspensions or dispersions described herein include, for example, potassium sorbate, parabens (e.g., methylparaben and propylparaben) and their salts, benzoic acid and its salts, other esters of para hydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

In one embodiment, the aqueous liquid dispersion can comprise methylparaben and propylparaben in a concentration ranging from about 0.01% to about 0.3% methylparaben by weight to the weight of the aqueous dispersion and about 0.005% to about 0.03% propylparaben by weight to the total aqueous dispersion weight. In yet another embodiment, the aqueous liquid dispersion can comprise methylparaben from about 0.05 to about 0.1 weight % and propylparaben from about 0.01 to about 0.02 weight % of the aqueous dispersion.

Suitable viscosity enhancing agents for the aqueous suspensions or dispersions described herein include, but are not limited to, methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans and combinations thereof. The concentration of the viscosity-enhancing agent will depend upon the agent selected and the viscosity desired.

In addition to the excipient listed above, the liquids of the present invention formulations may comprise inert diluents commonly used in the art, such as water or other solvents, solubilizing agents, emulsifiers, and/or sweeteners.

The formulations suitable for intramuscular, subcutaneous, or intravenous injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Additionally, the active agent(s) of the present invention can be dissolved at concentrations of >1 mg/ml using water-soluble beta cyclodextrins (e.g. beta-sulfobutyl-cyclodextrin and 2-hydroxypropylbetacyclodextrin. Proper fluidity can be maintained, for example, by the use of a coating such as a lecithin, by the maintenance of the required particle size in the case of dispersions, and by the use of surfactants. The active agent(s) of the present invention formulations suitable for subcutaneous injection may also contain excipients such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like. It may also be desirable to include isotonic agents, such as sugars, sodium chloride, and the like. Prolonged drug absorption of the injectable pharmaceutical form can be brought about by the use of agents delaying absorption, such as aluminum monostearate and gelatin. The active agent(s) of the present invention suspension formulations designed for extended-release via subcutaneous or intramuscular injection can avoid first-pass metabolism and lower dosages of the active agent(s) of the present invention will be necessary to maintain plasma levels of about 50 ng/ml. In such formulations, the particle size of the active agent(s) of the present invention particles and the range of the particle sizes of the active agent(s) of the present invention particles can be used to control the release of the drug by controlling the rate of dissolution in fat or muscle.

In still other embodiments, effervescent powders containing at least one co-crystal of the invention may be prepared. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the present invention are added to water, the acids and the base react to liberate carbon dioxide gas, thereby causing “effervescence.” Examples of effervescent salts include e.g: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In other embodiments, a powder comprising the polymorph(s) of the present invention may be formulated to comprise one or more pharmaceutical excipients and flavors. Such a powder may be prepared, for example, by mixing the active agent(s) of the present invention formulation and optional pharmaceutical excipients to form a bulk blend composition. Additional embodiments also comprise a suspending agent and/or a wetting agent. This bulk blend is uniformly subdivided into unit dosage packaging or multi-dosage packaging units. The term “uniform” means the homogeneity of the bulk blend is substantially maintained during the packaging process.

In certain embodiments of the present invention, pharmaceutical compositions may be formulated into a dosage form suitable for parenteral use. For example, the dosage form may be a lyophilized powder, a solution, suspension (e.g., depot suspension).

In other embodiments, pharmaceutical compositions may be formulated into a topical dosage form such as, but not limited to, a patch, a gel, a paste, a cream, an emulsion, liniment, balm, lotion, and ointment.

Tablets of the invention described here can be prepared by methods well known in the art. Various methods for the preparation of the immediate release, modified release, controlled release, and extended-release dosage forms (e.g., as matrix tablets, tablets having one or more modified, controlled, or extended-release layers, etc.) and the vehicles therein are well known in the art. Generally recognized compendium of methods include: Remington: The Science and Practice of Pharmacy, Alfonso R. Gennaro, Editor, 20th Edition, Lippincott Williams & Wilkins, Philadelphia, PA; Sheth et al. (1980) Compressed tablets, in Pharmaceutical dosage forms, Vol 1, edited by Lieberman and Lachtman, Dekker, NY.

In certain embodiments, solid dosage forms, e.g., tablets, effervescent tablets, and capsules, are prepared by mixing the active agent(s) of the present invention particles with one or more pharmaceutical excipients to form a bulk blend composition. When referring to these bulk blend compositions as homogeneous, it is meant that the active agent(s) of the present invention particles are dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluents. These the active agent(s) of the present invention formulations can be manufactured by conventional pharmaceutical techniques.

Conventional pharmaceutical techniques for preparation of solid dosage forms include, e.g., one or a combination of methods: (1) dry mixing, (2) direct compression, (3) milling, (4) dry or non-aqueous granulation, (5) wet granulation, or (6) fusion. See, e.g., Lachman et al., Theory and Practice of Industrial Pharmacy (1986). Other methods include, e.g., spray drying, pan coating, melt granulation, granulation, fluidized bed spray drying or coating (e.g., Wurster coating), tangential coating, top spraying, tableting, extruding and the like.

Compressed tablets are solid dosage forms prepared by compacting the bulk blend the active agent(s) of the present invention formulations described above. In various embodiments, compressed tablets which are designed to dissolve in the mouth will comprise one or more flavoring agents. In other embodiments, the compressed tablets will comprise a film surrounding the final compressed tablet. In some embodiments, the film coating can provide a delayed release of the active agent(s) of the present invention formulation. In other embodiments, the film coating aids in patient compliance (e.g., Opadry® coatings or sugar coating). Film coatings comprising Opadry® typically range from about 1% to about 3% of the tablet weight. Film coatings for delayed-release usually comprise 2-6% of a tablet weight or 7-15% of a spray-layered bead weight. In other embodiments, the compressed tablets comprise one or more excipients.

A capsule may be prepared, e.g., by placing the bulk blend of co-crystals of the present invention formulation, described above, inside of a capsule. In some embodiments, the co-crystals are placed in a soft gelatin capsule. In other embodiments, the co-crystals are placed in standard gelatin capsules or non-gelatin capsules such as capsules comprising HPMC. In other embodiments, the co-crystals of the present invention formulations are placed in a sprinkle capsule, wherein the capsule may be swallowed whole or the capsule may be opened and the contents sprinkled on food prior to eating. In some embodiments of the present invention, the therapeutic dose is split into multiple (e.g., two, three, or four) capsules. In some embodiments, the entire dose of the active agent(s) of the present invention formulation is delivered in a capsule form. For example, the capsule may comprise between about 100 mg to about 1000 mg of the active agent(s) of the present invention.

In certain preferred embodiments, the formulations of the present invention are fixed-dose combinations of the polymorph of the invention and at least one drug which can prevent, inhibit or treat a coronavirus infection in a human by a similar or different mechanism than the polymorph of the invention. Fixed-dose combination formulations may contain the following combinations in the form of single-layer monolithic tablet or multi-layered monolithic tablet or in the form of a core tablet-in-tablet or multi-layered multi-disk tablet or beads inside a capsule or tablets inside a capsule but not limited to: (a) therapeutically efficacious fixed-dose combinations of immediate-release formulations; (b) therapeutically efficacious fixed-dose combinations of immediate release and extended-release drugs contained in a single dosage form; (c) therapeutically efficacious fixed-dose combinations of extended-release formulations of the drug(s).

The pharmaceutical compositions described herein can be formulated into any suitable dosage form, including but not limited to, aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilized formulations, tablets, capsules, pills, powders, delayed-release formulations, immediate-release formulations, modified release formulations, extended-release formulations, pulsatile release formulations, multi particulate formulations, and mixed immediate release and controlled release formulations. In some embodiments, the co-crystals of the present invention formulations provide a therapeutically effective amount of the active agent(s) of the present invention over an interval of about 30 minutes to about 24 hours after administration, enabling, for example, a four times a day (Q.I.D.), a three times a day (t.i.d.), a twice-a-day (b.i.d.), or a once-a-day (q.d.) administration. The dosage form comprises co-crystals and a sufficient amount of a controlled release agent admixed with and/or coating the co-crystal to provide a desired in-vitro release profile and render the dosage form suitable, e.g., for a four time a day, a three times a day, a two times a day, or a once-a-day administration. In one embodiment, the co-crystals formulated into a controlled release or pulsatile solid dosage form for twice-a-day administration. In other embodiments, the co-crystals of the present invention are dispersed in aqueous dispersion for twice-a-day administration. Generally speaking, one will desire to administer an amount of the co-crystals of the present invention that is effective to achieve a plasma level commensurate with the concentrations found to be effective in vivo for a period of time effective to elicit a desired therapeutic effect.

Depending on the desired release profile, the oral solid dosage forms of the present invention may contain a suitable amount of controlled-release agents, extended-release agents, and/or modified-release agents (e.g., delayed-release agents). The pharmaceutical solid oral dosage forms comprising the active agent(s) of the present invention described herein can be further formulated to provide a modified or controlled release of the active agent(s) of the present invention. In some embodiments, the solid dosage forms described herein can be formulated as a delayed release dosage form such as and enteric-coated delayed release oral dosage forms, i.e., as an oral dosage form of a pharmaceutical composition as described herein which utilizes an enteric coating to affect release in the small intestine of the gastrointestinal tract. The enteric-coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated. Enteric coatings may also be used to prepare other controlled release dosage forms including extended-release and pulsatile release dosage forms.

In other embodiments, the active agent(s) of the formulations described herein are delivered using a pulsatile dosage form. Pulsatile dosage forms comprising the active agent(s) of the present invention formulations described herein may be administered using a variety of formulations known in the art. For example, such formulations include, but are not limited to, those described in U.S. Pat. Nos. 5,011,692, 5,017,381, 5,229,135, and 5,840,329, each of which is specifically incorporated by reference. Other dosage forms suitable for use with the active agent(s) of the present invention formulations are described in, for example, U.S. Pat. Nos. 4,871,549, 5,260,068, 5,260,069, 5,508,040, 5,567,441 and 5,837,284, all of which are specifically incorporated by reference. In one embodiment, the controlled release dosage form is pulsatile release solid oral dosage form comprising at least two groups of particles, each containing active agent(s) of the present invention as described herein. The first group of particles provides a substantially immediate dose of the active agent(s) of the present invention upon ingestion by a subject. The first group of particles can be either uncoated or comprise a coating and/or sealant. The second group of particles comprises coated particles, which may comprise from about 2% to about 75%, preferably from about 2.5% to about 70%, or from about 40% to about 70%, by weight of the total dose of the active agent(s) of the present invention in said formulation, in admixture with one or more binders.

Coatings for providing a controlled, delayed, or extended-release may be applied to the drug(s) or to a core containing the drug(s). The coating may comprise a pharmaceutically acceptable ingredient in an amount sufficient, e.g., to provide a delay of from about 2 hours to about 7 hours following ingestion before release of the second dose. Suitable coatings include one or more differentially degradable coatings such as, by way of example only, pH-sensitive coatings (enteric coatings) such as acrylic resins (e.g., Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, and Eudragit® NE30D, Eudragit® NE 40DR) either alone or blended with cellulose derivatives, e.g., ethylcellulose, or non-enteric coatings having variable thickness to provide differential release of the active agent(s) of the present invention formulation.

Many other types of controlled/delayed/extended-release systems known to those of ordinary skill in the art and are suitable for use with the active agent(s) of the present invention formulations described herein. Examples of such delivery systems include, e.g., polymer-based systems, such as polylactic and polyglycolic acid, polyanhydrides and polycaprolactone, cellulose derivatives (e.g., ethylcellulose), porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2nd Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983, each of which is specifically incorporated by reference. In certain embodiments, the controlled release systems may comprise the controlled/delayed/extended-release material incorporated with the drug(s) into a matrix, whereas in other formulations, the controlled release material may be applied to a core containing the drug(s). In certain embodiments, one drug may be incorporated into the core while the other drug is incorporated into the coating. In some embodiments, materials include shellac, acrylic polymers, cellulosic derivatives, polyvinyl acetate phthalate, and mixtures thereof. In other embodiments, materials include Eudragit® series E, L, RL, RS, NE, L, L300, S, 100-55, cellulose acetate phthalate, Aquateric, cellulose acetate trimellitate, ethyl cellulose, hydroxypropylmethylcellulose phthalate, hydroxypropylmethylcellulose acetate succinate, polyvinyl acetate phthalate, and Cotteric. The controlled/delayed/extended-release systems may utilize a hydrophilic polymer, including but not limited to a water-swellable polymer (e.g., a natural or synthetic gum). The hydrophilic polymer may be any pharmaceutically acceptable polymer which swells and expands in the presence of water to slowly release the active agent(s) of the present invention. These polymers include polyethylene oxide, methylcellulose, hydroxypropyl cellulose, hydroxypropylmethylcellulose, and the like.

The performance of acrylic polymers (primarily their solubility in biological fluids) can vary based on the degree and type of substitution. Examples of suitable acrylic polymers which may be used in matrix formulations or coatings include methacrylic acid copolymers and ammonia methacrylate copolymers. The Eudragit series E, L, S, RL, RS and NE (Rohm Pharma) are available as solubilized in an organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in the stomach and dissolve in the intestine; Opadry Enteric is also insoluble in the stomach and dissolves in the intestine.

Examples of suitable cellulose derivatives for use in matrix formulations or coatings include ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous-based system and is a spray-dried CAP psuedolatex with particles <1 μm. Other components in Aquateric can include pluronic, Tweens, and acetylated monoglycerides. Other suitable cellulose derivatives include cellulose acetate trimellitate (Eastman); methylcellulose (Pharmacoat, Methocel); hydroxypropylmethylcellulose phthalate (HPMCP); hydroxypropylmethylcellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules or as fine powders for aqueous dispersions. Other suitable cellulose derivatives include hydroxypropylmethylcellulose.

In some embodiments, the coating may contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate, and triacetin. Conventional coating techniques such as spray or pan coating are employed to apply coatings. The coating thickness must be sufficient to ensure that the oral dosage form remains intact until the desired site of topical delivery in the intestinal tract is reached.

Extended-release multi-layered matrix tablets may be prepared by using fixed-dose combinations of a drug(s) from Group 1 together with a drug(s) from Group 2. Such formulations may comprise one or more of the drugs within a hydrophilic or hydrophobic polymer matrix. For example, a hydrophilic polymer may comprise guar gum, hydroxypropylmethylcellulose, and xanthan gum as matrix formers. Lubricated formulations may be compressed by a wet granulation method.

Multilayer tablet delivery (e.g., such as that used in the GeoMatrix™ technology) comprises a hydrophilic matrix core containing the active ingredient and one or two impermeable or semi-permeable polymeric coatings. This technology uses films or compressed polymeric barrier coatings on one or both sides of the core. The presence of polymeric coatings (e.g., such as that used in the GeoMatrix™ technology) modifies the hydration/swelling rates of the core and reduces the surface area available for drug release. These partial coatings provide modulation of the drug dissolution profile: they reduce the release rate from the device and shift the typical time-dependent release rate towards constant release. This technology enables customized levels of controlled release of specific drugs and/or simultaneous release of two different drugs at different rates that can be achieved from a single tablet. The combination of layers, each with different rates of swelling, gelling and erosion, is used for the rate of drug release in the body. Exposure of the multilayer tablet as a result of the partial coating may affect the release and erosion rates, therefore, transformation of a multilayered tablet with exposure on all sides to the gastrointestinal fluids upon detachment of the barrier layer will be considered.

Multi-layered tablets containing combinations of immediate release and modified/extended release of two different drugs or dual release rate of the same drug in a single dosage form may be prepared by using hydrophilic and hydrophobic polymer matrices.

Dual release repeat action multi-layered tablets may be prepared with an outer compression layer with an initial dose of rapidly disintegrating matrix in the stomach and a core inner layer tablet formulated with components that are insoluble in the gastric media but release efficiently in the intestinal environment.

In one embodiment, the dosage form is a solid oral dosage form which is an immediate release dosage form whereby >80% of the active agent(s) of the present invention particles hours after administration. In other embodiments, the invention provides an (e.g., solid oral) dosage form that is a controlled release or pulsatile release dosage form. In such instances, the release may be, e.g., 30 to 60% of the active agent(s) of the present invention particles by weight are released from the dosage form within about 2 hours after administration and about 90% by weight of the active agent(s) of the present invention released from the dosage form, e.g., within about 7 hours after administration. In yet other embodiments, the dosage form includes at least one active agent in an immediate-release form and at least one active agent in the delayed-release form, or sustained-release form. In yet other embodiments, the dosage form includes at least two active agents that are released at different rates as determined by in-vitro dissolution testing or via oral administration.

The various release dosage formulations discussed above and others known to those skilled in the art can be characterized by their disintegration profile. A profile is characterized by the test conditions selected. Thus the disintegration profile can be generated at a pre-selected apparatus type, shaft speed, temperature, volume, and pH of the dispersion media. Several disintegration profiles can be obtained. For example, a first disintegration profile can be measured at a pH level approximating that of the stomach (about pH 1.2); a second disintegration profile can be measured at a pH level approximating that of one point in the intestine or several pH levels approximating multiple points in the intestine (about 6.0 to about 7.5, more specifically, about 6.5 to 7.0). Another disintegration profile can be measured using distilled water. The release of formulations may also be characterized by their pharmacokinetic parameters, for example, Cmax, Tmax, and AUC (0-τ).

In certain embodiments, the controlled, delayed or extended-release of one or more of the drugs of the fixed-dose combinations of the invention may be in the form of a capsule having a shell comprising the material of the rate-limiting membrane, including any of the coating materials previously discussed, and filled with the active agent(s) of the present invention particles. A particular advantage of this configuration is that the capsule may be prepared independently of the active agent(s) of the present invention particles; thus process conditions that would adversely affect the drug can be used to prepare the capsule. Alternatively, the formulation may comprise a capsule having a shell made of a porous or a pH-sensitive polymer made by a thermal forming process. Another alternative is a capsule shell in the form of an asymmetric membrane; i.e., a membrane that has a thin skin on one surface and most of whose thickness is constituted of a highly permeable porous material. The asymmetric membrane capsules may be prepared by a solvent exchange phase inversion, wherein a solution of polymer, coated on a capsule-shaped mold, is induced to phase-separate by exchanging the solvent with a miscible non-solvent. In another embodiment, spray layered active agent(s) of the present invention particles are filled in a capsule. An exemplary process for manufacturing the spray layered the active agent(s) of the present invention is the fluidized bed spraying process. The active agent(s) of the present invention suspensions or the active agent(s) of the present invention complex suspensions described above may be sprayed onto sugar or microcrystalline cellulose (MCC) beads (20-35 mesh) with Wurster column insert at an inlet temperature of 50° C. to 60° C. and air temp of 30° C. to 50° C. A 15 to 20 wt % total solids content suspension containing 45 to 80 wt % the active agent(s) of the present invention, 10 to 25 wt % hydroxymethylpropylcellulose, 0.25 to 2 wt % of SLS, 10 to 18 wt % of sucrose, 0.01 to 0.3 wt % simethicone emulsion (30% emulsion) and 0.3 to 10% NaCl, based on the total weight of the solid content of the suspension, are sprayed (bottom spray) onto the beads through 1.2 mm nozzles at 10 mL/min and 1.5 bar of pressure until a layering of 400 to 700% wt % is achieved as compared to initial beads weight. The resulting spray layered the active agent(s) of the present invention particles or the active agent(s) of the present invention complex particles comprise about 30 to 70 wt % of the active agent(s) of the present invention based on the total weight of the particles. In one embodiment the capsule is a size 0 soft gelatin capsule. In one embodiment, the capsule is a swelling plug device. In another embodiment, the swelling plug device is further coated with cellulose acetate phthalate or copolymers of methacrylic acid and methylmethacrylate. In some embodiments, the capsule includes at least 100 mg (or at least 300 mg or at least 400 mg) the active agent(s) of the present invention and has a total weight of less than 800 mg (or less than 700 mg). The capsule may contain a plurality of the active agent(s) of the present invention-containing beads, for example, spray layered beads. In some embodiments, the beads are 12-25% the active agent(s) of the present invention by weight. In some embodiments, some or all of the active agent(s) of the present invention containing beads are coated with a coating comprising 6 to 15% (or 8 to 12%) of the total bead weight. Optimization work typically involves lower loading levels and the beads constitute 30 to 60% of the finished bead weight. The capsule may contain a granulated composition, wherein the granulated composition comprises the active agent(s) of the present invention.

The capsule may provide pulsatile release the active agent(s) of the present invention oral dosage form. Such formulations may comprise: (a) a first dosage unit comprising a first the active agent(s) of the present invention dose that is released substantially immediately following oral administration of the dosage form to a patient; (b) a second dosage unit comprising a second the active agent(s) of the present invention dose that is released approximately 3 to 7 hours following administration of the dosage form to a patient. For pulsatile release capsules containing beads, the beads can be coated with a coating comprising 6 to 15% (or 8 to 12%) of the total bead weight. In some embodiments, the coating is a coating that is insoluble at pH 1 to 2 and soluble at pH greater than 5.5. In certain embodiments, the formulation may comprise a pulsatile release capsule comprising at least two active agents (e.g., one drug from Group 1 and one drug from Group 2). This pulsatile release capsule may contain a plurality of beads in which some beads are immediate-release beads and other beads are formulated, for example with the use of a coating, for modified release, typically from about 3 to about 10 hours after administration. In other embodiments, the pulsatile release capsule contains a plurality of beads formulated for modified release and the active agent(s) of the present invention powder, for example, spray granulated the active agent(s) of the present invention, for immediate release.

In some embodiments, the release of the active agent(s) of the present invention particles can be modified with a modified release coating, such as an enteric coating using cellulose acetate phthalate or a sustained release coating comprising copolymers of methacrylic acid and methylmethacrylate. In one embodiment, the enteric coating may be present in an amount of about 0.5 to about 15 wt %, more specifically, about 8 to about 12 wt %, based on the weight of, e.g., the spray layered particles. In one embodiment, the spray layered particles coated with the delayed and/or sustained release coatings can be filled in a modified release capsule in which both enteric-coated and immediate release the active agent(s) of the present invention beads are filled into a soft gelatin capsule. Additional suitable excipients may also be filled with the coated particles in the capsule. The uncoated particles release the active agent(s) of the present invention immediately upon administration while the coated particles do not release the active agent(s) of the present invention until these particles reach the intestine. By controlling the ratios of the coated and uncoated particles, desirable pulsatile release profiles may be obtained. In some embodiments, the ratios between the uncoated and the coated particles are e.g., 20/80, or 30/70, or 40/60, or 50/50, w/w to obtain desirable release.

In certain embodiments, the drugs contained in a fixed-dose combination of the present invention may be in the form of beads contained within a capsule. In certain embodiments, some beads may release one or both drugs immediately, while other beads would release one or both drugs over an extended period of time or after a delay (delayed-release).

In certain embodiments, spray layered active agent(s) of the present invention particles can be compressed into tablets with commonly used pharmaceutical excipients. Any appropriate apparatus for forming the coating can be used to make the enteric coated tablets, e.g., fluidized bed coating using a Wurster column, powder layering in coating pans or rotary coaters; dry coating by double compression technique; tablet coating by film coating technique, and the like. See, e.g., U.S. Pat. No. 5,322,655; Remington's Pharmaceutical Sciences Handbook: Chapter 90 “Coating of Pharmaceutical Dosage Forms”, 1990. In certain embodiments, the spray layered the active agent(s) of the present invention described above and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the active agent(s) of the present invention formulation into the gastrointestinal fluid. In other embodiments, the spray layered the active agent(s) of the present invention particles or spray layered the active agent(s) of the present invention complex particles with enteric coatings described above and one or more excipients are dry blended and compressed into a mass, such as a tablet. In one embodiment, the enteric-coated particles in the tablet substantially avoid the release of the active agent(s) of the present invention, for example, less than 15 wt %, in the stomach but releases substantially all the active agent(s) of the present invention (enterically or sustained-release coated), for example, greater than 80 wt %, in the intestine.

In certain embodiments, a pulsatile release the active agent(s) of the present invention formulation comprises a first dosage unit comprising a formulation made from the active agent(s) of the present invention containing granules made from a spray drying or spray granulated procedure or a formulation made from the active agent(s) of the present invention complex containing granules made from a spray drying or spray granulated procedure without enteric or sustained-release coatings and a second dosage unit comprising spray layered the active agent(s) of the present invention particles or spray layered the active agent(s) of the present invention complex particles with enteric or sustained-release coatings. In one embodiment, the first dosage unit and the second dosage unit are wet or dry blended and compressed into a mass to make a pulsatile release tablet.

In certain embodiments, binding, lubricating and disintegrating agents are blended (wet or dry) to the spray layered the active agent(s) of the present invention to make a compressible blend. The first and second dosage units are compressed separately and then compressed together to form a bilayer tablet. In yet another embodiment, the first dosage unit is in the form of an overcoat and completely covers the second dosage unit.

In certain embodiments, ingredients (including or not including the active agent(s)) of the invention are wet granulated. The individual steps in the wet granulation process of tablet preparation include milling and sieving of the ingredients, dry powder mixing, wet massing, granulation, drying, and final grinding. In various embodiments, the active agent(s) of the present invention composition is added to the other excipients of the pharmaceutical formulation after they have been wet granulated. Alternatively, the ingredients may be subjected to dry granulation, e.g., via compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). No wet binder or moisture is involved in any of the steps. In some embodiments, the active agent(s) of the present invention formulation is dry granulated with other excipients in the pharmaceutical formulation. In other embodiments, the active agent(s) of the present invention formulation is added to other excipients of the pharmaceutical formulation after they have been dry granulated.

In other embodiments, the formulation of the present invention formulations described herein is a solid dispersion. Methods of producing such solid dispersions are known in the art and include, but are not limited to, for example, U.S. Pat. Nos. 4,343,789, 5,340,591, 5,456,923, 5,700,485, 5,723,269, and U.S. Pub. Appl. 2004/0013734, each of which is specifically incorporated by reference. In some embodiments, the solid dispersions of the invention comprise both amorphous and non-amorphous the active agent(s) of the present invention and can have enhanced bioavailability as compared to conventional the active agent(s) of the present invention formulations. In still other embodiments, the active agent(s) of the present invention formulations described herein are solid solutions. Solid solutions incorporate a substance together with the active agent and other excipients such that heating the mixture results in the dissolution of the drug and the resulting composition is then cooled to provide a solid blend that can be further formulated or directly added to a capsule or compressed into a tablet.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration.

The formulations and dosage forms are suitable for administration anywhere from 1 to 4 times per day.

### Methods of Treatment

The amyloid precursor protein (APP) is a broadly expressed transmembrane protein. APP is expressed, for example, in neurons, astrocytes and microglia. APP expression and its metabolism changes under various neuropathological conditions, especially in response to oxidative stress.

Cleavage of APP by gamma and beta secretases acting sequentially, generates a series of fragments including, e.g., amyloid β (Aβ) peptides and APP intracellular domain (AICD).

AICD is a transcriptional modulator that has been implicated in various physiological processes, including synaptic plasticity and cytoskeletal organization. However, under conditions of severe oxidative stress, AICD interacts with a transcriptional co-activator FOXO3a, to promote cell death. Additionally, AICD and FOXO3a have been shown to jointly control mitochondrial function by modulating PTEN induced putative kinase 1 (PINK1) transcription and to control the expression of Leucine Rich Repeat Kinase 2 (LRRK2). LRRK2 mutations adversely impair multiple physiological processes, including synaptic activity and plasticity, maintenance of normal dendritic spine morphology. Several lines of evidence indicate that overactive LRRK2 interferes with autophagic processes, including mitophagy. Based on these properties, inhibition of AICD activity is therefore anticipated to have beneficial effects in treatment of a number of diseases.

Although, Aβ accumulation has received the most attention, AICD which is produced concomitantly, is increasingly recognized as a likely major contributor to the pathogenesis of Alzheimer's disease (AD) and numerous other neurodegenerative disorders, e.g., Parkinson's disease (PD), Multiple Sclerosis (MS), juvenile neuronal ceroid lipofuscinosis (JNCL) (Batten disease type-3), age-related macular degeneration (AMD); Amylolateral sclerosis (ALS), mild cognitive impairment (MCI), neurologic injury (Traumatic Brain Injury (TBI)) and neurologic inflammation.

As explained above, bind the amyloid precursor protein (APP) and inhibit the transcriptional activity of its intracellular domain (AICD). Itanapraced may also modulate microglia. Thus, the polymorphs and crystalline systems of the present invention may utility in treatment of neurodegenerative disorders.

A method of treating a neurodegeneration disorder in a subject in need thereof in accordance with the present invention comprises administering to the subject a therapeutically effective amount of a polymorph of a co-crystal of itanapraced and nicotinamide. In one embodiment, the polymorph has an X-ray Powder Diffraction Pattern (XRPD) that is substantially the same as the XRPD shown in FIG. 8.

A method of treating a neurodegeneration disorder in a subject in need thereof in accordance with the present invention also comprises administering to the subject or a therapeutically effective amount of a crystalline system containing itanapraced and nicotinamide in a stoichiometric ratio of about 1:1, wherein itanapraced and nicotinamide are linked by hydrogen bonding and other non-covalent and non-ionic interactions, the crystalline system is solid at 25° C. and has an X-ray Powder Diffraction Pattern (XRPD) that is substantially the same as the XRPD shown in FIG. 8.

### Dosage

A polymorph of a crystal of itanapraced and nicotinamide may be administered to a subject at a dose of a dose of about 3 mg/day to about 3000 mg/day, about 4 mg/day to about 2500 mg/day, about 5 mg/day to about 2000 mg/day, about 10 mg/day to about 1500 mg/day, 10 mg/day to about 1000 mg/day, about 50 mg/day to about 600 mg/day, about 50 mg/day to about 500 mg/day, about 50 mg/day to about 400 mg/day, 50 mg/day to about 300 mg/day, or about 100 mg/day to about 30 mg/day.

The crystalline system containing itanapraced and nicotinamide in a stoichiometric ratio of about 1:1, wherein itanapraced and nicotinamide are linked by hydrogen bonding and other non-covalent and non-ionic interactions, the crystalline system is solid at 25° C., may also be administered to a subject at a dose of a dose of about 3 mg/day to about 3000 mg/day, about 4 mg/day to about 2500 mg/day, about 5 mg/day to about 2000 mg/day, about 10 mg/day to about 1500 mg/day, 10 mg/day to about 1000 mg/day, about 50 mg/day to about 600 mg/day, about 50 mg/day to about 500 mg/day, about 50 mg/day to about 400 mg/day, 50 mg/day to about 300 mg/day, or about 100 mg/day to about 30 mg/day.

### Administration

The formulations of the present invention may be administered by any pharmaceutically effective route. For example, the polymorph(s) and the crystalline system(s) of the invention may be formulated in a manner such that they can be administered orally, intranasally, rectally, vaginally, sublingually, buccally, parenterally, or transdermally, and thus, be formulated accordingly. The polymorph(s) and the crystalline system(s) of the invention can be administered in liquid, tablet, parenteral, transrectal, transdermal or in any other form of administration suitable in order to achieve a therapeutic effect. Such formulations may contain additional fillers, carriers, excipient or excipients, inert or not, known to those skilled in the art of pharmaceutical preparations, in order to provide appropriate volume and/or facilitate absorption of the polymorph(s) and the crystalline system(s).

In the methods of the invention comprising oral administration, the formulations and dosage forms may be administered anywhere from 1 to 4 times per day, in order to provide the full daily dose.

Different embodiments of the invention include, but are not limited to, the following examples. In certain embodiments, the polymorph(s) and the crystalline system(s) of the invention is (are) administered together or separately but concurrently with an additional drug which may work via the same or different mechanism to prevent, inhibit or treat infection by a neurodegenerative disorder. Another embodiment of the invention includes multiple variations in the pharmaceutical dosages of each drug in combination in a single dosage form as further outlined below. Another embodiment of the invention includes various forms of preparations including using solids, liquids, immediate or delayed or extended-release forms. Many types of variations are possible as known to those skilled in the art. Another embodiment of the invention includes multiple routes of administration, which may differ in different patients according to their preference, co-morbidities, side effect profile, and other factors (IV, PO, transdermal, etc.). Another embodiment of the invention includes the presence of other substances with the polymorph(s) and the crystalline system(s) of the invention, known to those skilled in the art, such as fillers, carriers, gels, skin patches, lozenges or other modifications in the preparation to facilitate absorption through various routes (such as gastrointestinal, transdermal, etc.) and/or to extend the effect of the drugs, and/or to attain higher or more stable serum levels or to enhance the therapeutic effect of the polymorph(s) and the crystalline system(s) of the invention.

### EXAMPLE 1

CSPNCT1 and CSPNCT2 were formed and identified, and two processes to synthesize CSPNCT2 were developed, one in DCM and the other in Ethyl Acetate. Both processes have shown reproducibility and scalability at gram scale. The experimental work summarized in Table 3.

TABLE 1

| Notebook | Input | Output | Comments |
|---|---|---|---|
| R26323-026 | 100 mg | NA | Solubility Study |
| R26323-027 | 150 mg | NA | Co-Crystal Pilots |
| R26323-028 | 200 mg | NA | Co-Crystal Pilots |
| | | | (Methanol) |
| R26323-029 | 100 mg | NA | Solubility Study |
| | | | (NCT) |
| R26323-030 | 200 mg | NA | DCM-Heptane |
| | | | Crystallization |
| R26323-031 | 200 mg | NA | CSP-1103 |
| | | | Recrystallization in |
| | | | DCM |
| R26323-032 | 300 mg | NA | Co-Crystal pilots |
| | | | cont. |
| R26323-033 | 4.0 g | 2.7 g | CSPNCT2 DCM |
| | | | scale up |
| R26323-034 | 5.0 g | 4.1 g | CSPNCT2 Ethyl |
| | | | Acetate scale up |

### EXAMPLE 2

### Solubility Studies

Solubility data was collected on both Nicotinamide and CSP-1103 over a range of solvents. This was accomplished by saturating each solvent with the respective substrate (Nicotinamide or CSP-1103) at 20-25° C., filtering off the solid, and then sampling the supernatant by High Performance Liquid Chromatography (HPLC) to determine an area response. The subsequent response was compared to the response factor of a known standard to determine the concentration of the substrate dissolved in solution at the saturation point. In cases where the solubility was greater than 100 mg/mL in the target solvent, it was considered highly soluble and listed as “>100 mg/mL”.

The solubility studies were run in experiment R26323-026 and results are listed in Table 4 below. The target of this experiment was to find congruent or incongruent solubilities between Nicotinamide or CSP-1103 to design a crystallization workspace that would encourage co-crystallization. Some solubilities are listed as general ranges (i.e. >100 mg/mL or <20 mg/mL), as the target was relative overlapping or opposite solubility characteristics. Based on these results, Ethyl Acetate, 2-butanol, Toluene, and dichloromethane were all determined to be within approximately 20 mg/mL solubility between the two co-formers. Acetonitrile was also identified, but not attempted in downstream pilots. Methanol was identified as a solvent with incongruent solubility, as Nicotinamide was highly soluble at >100 mg/mL while CSP-1103 had relatively low solubility at 11 mg/mL.

TABLE 2
Solubility Table

| | CSP-1103 | NCT | |
|---|---|---|---|
| | Solubility | Solubility | |
| Solvent | (mg/mL) | (mg/mL) | Comment |
| Ethyl Acetate | 21 | 12 | ≤20 mg/mL difference |
| (EtOAc) | | | in solubility |
| Dimethyl Sulfoxide | >100 | >100 | |
| (DMSO) | | | |
| Tert-butyl methyl | 22 | 2 | |
| ether (MTBE) | | | |
| 2-butanol | 11 | 31 | ≤20 mg/mL difference |
| | | | in solubility |
| Methanol (MeOH) | 11 | 200 | High NCT, solubility, |
| | | | low CSP-1103 |
| | | | solubility |
| Toluene | 3 | <20 | ≤20 mg/mL difference |
| | | | in solubility |
| 2-Me THF | >100 | 10 | |
| Dichloromethane | 7 | <20 | ≤20 mg/mL difference |
| (DCM) | | | in solubility |
| Acetonitrile (ACN) | 3 | 15 | ≤20 mg/mL difference |
| | | | in solubility |
| n-heptane | <1 | <1 | Possible anti-solvent |
| Ethanol (EtOH) | 15 | 100 | |
| Isopropanol (IPA) | 10 | 60 | |

### EXAMPLE 3

### Crystallization Pilots (Screening)

Based on R26323-022, CSP1103NCT co-crystals were formed by mixing a saturated solution of NCT (at 10 equivalents excess) in Ethyl Acetate with 1 equivalent of CSP-1103 and then using rapid evaporation until solids precipitated. In this case, CSPNCT1 was observed, consistent with the polymorph identified by solid state co-crystal screening. While this provided the target co-crystals, there was also significant nicotinamide (NCT) present, likely due to the lack of control when evaporating solvent and the excess of NCT used. These experiments also showed low reproducibility (which can be observed in R26323-023 and R26323-025). Despite this, the experiments revealed that a co-crystal can be formed using techniques like solvent evaporation.

Using previous experiments and collected solubility data, a set of pilot experiments were set up using the following procedure (R26323-027):
1. Dissolve 1 equivalent of CSP-1103 in target solvent (Ethyl Acetate, 2-butanol, DCM, Toluene);
2. Dissolve 1 equivalent of NCT in the same volume of same target solvent;
3. Combine the solutions with stirring at 50° C.;
4. Allow the solution to cool to ambient temperature (20-25° C.); and
5. Split the solution into two aliquots.

Splitting the resulting 1:1 stoichiometric mixture of CSP-1103 and NCT into 2 aliquots allows for testing two methods of crystallization for each solvent. With one aliquot, neat crystallization was attempted. If no solids crystallized over time, solvent was blown off using a nitrogen stream until solids precipitated. With the other aliquot, heptane was added as an anti-solvent dropwise until solids precipitated. In both cases, when solids were achieved, the resulting slurries were filtered, and the solids were dried using a vacuum. All the pilots in this round were run at 100 mg scale.

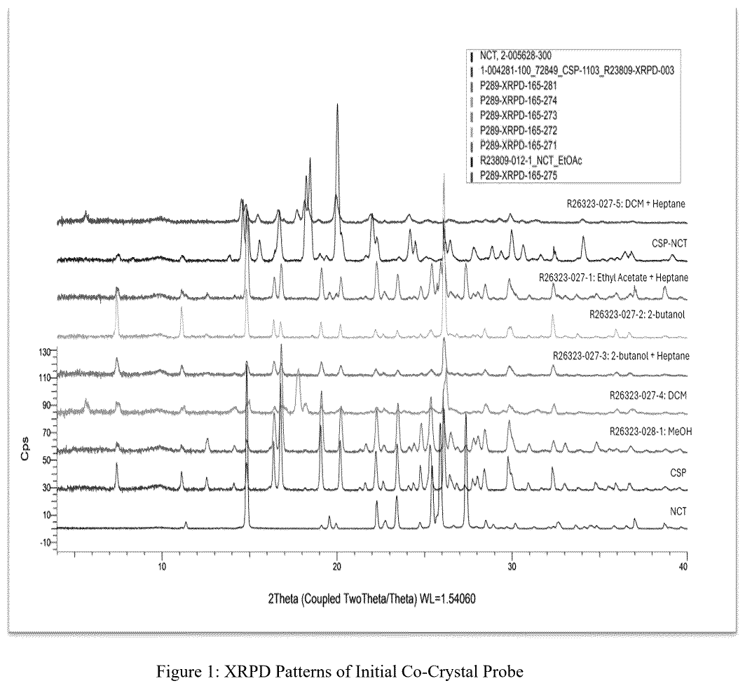

The results of these initial pilots are listed in Table 5 below. Each aliquot can be identified by the neat solvent or the target solvent with Heptane as an anti-solvent. The success of forming the desired co-crystal identified during solid state work was tracked using XRPD (X-ray powder diffraction). The resulting solid XRPD pattern was compared qualitatively to the pattern of NCT, CSP-1103, and the single crystal data collected of CSPNCT1 during solid state work. Results of neat Ethyl Acetate were not applicable due to a lack of solids forming. The XRPD patterns for these experiments can be observed in FIG. 1. Results of the toluene experiment were not applicable due to the exorbitant volume of toluene needed to dissolve the co-formers, making it an unrealistic approach to forming co-crystal (this experiment was discarded before splitting into aliquots).

XRPD results indicated two potential hits. The experiment in dichloromethane where heptane was added as an anti-solvent (R26323-027-5) exhibited a pattern consistent with the CSPNCT1 co-crystal identified during solid state screening. There were also signals that were not consistent with either CSP-1103, NCT, or the previously observed CSPNCT1 co-crystal. No patterns indicated the presence of significant levels of either individual co-former. The XRPD indicated low crystallinity. While the experiment in neat dichloromethane (R26323-027-4) did not show a pattern consistent with the desired co-crystal CSPNCT1, it exhibited the same pattern seen in the DCM/heptane experiment that was not consistent with either individual co-former. It also contained a pattern consistent with CSP-1103.

Because the new pattern was observed solely in experiments using dichloromethane, some CSP-1103 was recrystallized in DCM to rule out the potential of a DCM related polymorph or solvate (R26323-031-1). The recrystallized material had an XRPD pattern consistent with that of CSP-1103, indicating that it was unlikely the new pattern was the result of a polymorph or solvate relating to DCM. This evidence indicated high likelihood that there was a newly observed polymorph of the CSPNCT co-crystal being formed. The co-crystal previously identified by solid state work (and observed as a mixture in R26323-027-5) was identified as CSPNCT1, or co-crystal Form 1, while co-crystal consistent with the newly identified pattern was identified as CSPNCT2, or co-crystal Form 2. Analysis by XRPD of R26323-027-5 appears as a mixture of CSPNCT1 and CSPNCT2.

The experiment with DCM and heptane was repeated in R26323-030-1 (as well as the experiment with Ethyl Acetate and heptane in R26323-030-2), but these experiments showed no co-crystal present of either potential form with signals largely consistent with CSP-1103. These results were emblematic of previous issues with reproducibility in forming co-crystal material.

Per Table 5 below, there was also an extraneous experiment using a slightly different workspace in Methanol. Methanol was the only target solvent attempted with an incongruent solubility between both co-formers. In this case, Nicotinamide was highly soluble in Methanol whereas CSP-1103 had low solubility. Methanol was charged to CSP-1103 at 55° C. until the CSP-1103 was in solution. NCT was then added as a solid to the solution with stirring. The resulting solution was then allowed to cool to ambient temperature. No precipitate formed upon cooling to ambient temperature (20-25° C.), indicating the possibility that Nicotinamide and CSP-1103 have a higher coordinating solubility than CSP-1103 has when dissolved in an isolated system. After stirring overnight, a white precipitate had formed. The solids were filtered, dried, and run by XRPD. XRPD results showed no signal consistent with co-crystal and results consistent with CSP-1103.

TABLE 3
Crystallization Pilots (Initial Probe)

| | | | XRPD co- |
|---|---|---|---|
| Pilot | Qualitative Comments | Lot # | crystal |
| Ethyl Acetate | Solvent reduced, small | NA | NA |
| | unfilterable crystals | | |
| Ethyl | Solid precipitation upon | R26323- | Mostly CSP- |
| Acetate/Heptane | addition of heptane | 027-1 | 1103 |
| 2-Butanol | Solid precipitation upon | R26323- | Mostly CSP- |
| | reduced solvent | 027-2 | 1103 |
| 2-Butanol/ | Solid precipitation upon | R26323- | Mostly CSP- |
| Heptane | reduced solvent | 027-3 | 1103 |
| Toluene | Not viable | NA | NA |
| DCM | Solid precipitation upon | R26323- | CSPNCT2 |
| | reduced solvent | 027-4 | |
| DCM/Heptane | Solid precipitation upon | R26323- | CSPNCT1 and |
| | addition of heptane | 027-5 | CSPNCT2 |
| Methanol | Precipitation upon | R26323- | Mostly CSP- |
| | cooling to ambient | 028-1 | 1103 |
| | temperature and stirring | | |
| DCM/Heptane | Precipitation in similar | R26323- | Mostly CSP- |
| (Repeat | fashion to original pilot | 030-1 | 1103 |
| procedure) | | | |
| Ethyl | Precipitation in similar | R26323- | Mostly CSP- |
| Acetate/Heptane | fashion to original pilot | 030-2 | 1103 |

With inconsistency in repeated experiments, but the ability to form two potential CSPNCT co-crystal polymorphs, other techniques were attempted. Two experiments were set up to test the effect of seeding co-crystal material in a 1:1 stoichiometric mixture of CSP-1103 and NCT (R26323-032-1 and R26323-032-2). The experiments were differentiated by the solvent used with one in Ethyl Acetate and the other in DCM. The experiments were run at 200-300 mg scale:
1. Charge 1 equivalent of NCT and 1 equivalent of CSP-1103;
2. Charge target solvent (EtOAc or DCM);
3. Heat to 50° C. to fully dissolve;
4. Cool to ambient temperature (20-25° C.);
5. Seed with R26323-027-5 (mixture of Form 1 and Form 2); and
6. Filter precipitate.

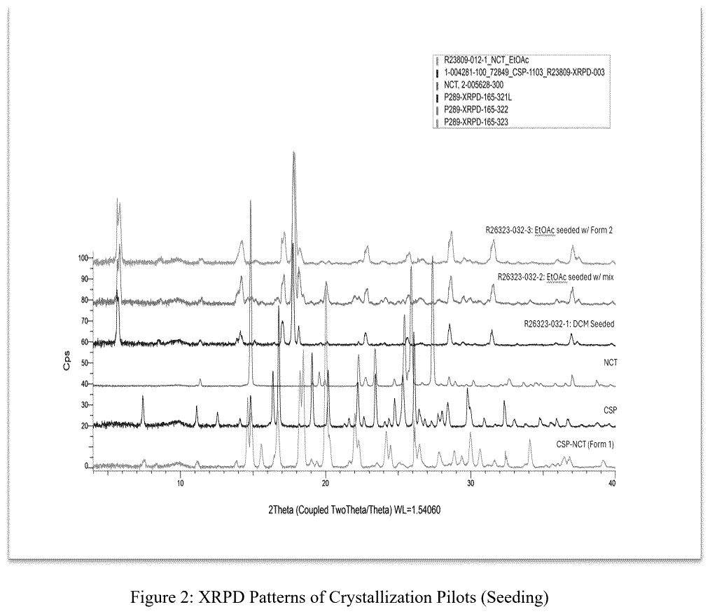

The results can be observed in Table 6 below. XRPD patterns for the seeding experiments can be observed in FIG. 2 below. Interestingly, when the experiment was run in DCM and seeded with the mixture of co-crystal polymorphs, the resulting filtered solids had an XRPD pattern consistent exclusively with Form 2, or CSPNCT2 (R26323-032-1). No pattern consistent with individual crystalline CSP-1103 or NCT was observed. When a similar experiment was run in Ethyl Acetate and seeded with the same material, the resulting solids had an XRPD pattern consistent with a mixture of both Form 1 and Form 2.

TABLE 4
Crystallization Pilots (Seeding)

| | Qualitative | | XRPD co- |
|---|---|---|---|
| Pilot | Comments | Lot # | crystal |
| DCM - Seeded with | Seeded with mixed | R26323- | CSPNCT2 |
| R26323-027-5 | co-crystal | 032-1 | |
| Ethyl Acetate - | Seeded with mixed | R26323- | CSPNCT1 and |
| Seeded with R26323- | co-crystal | 032-2 | CSPNCT2 |
| 027-5 | | | |
| Ethyl Acetate - | Seeded with | R26323- | CSPNCT2 |
| Seeded R26323-032-1 | CSPNCT2 co- | 032-3 | |
| | crystal | | |

After generating a lot of clean CSPNCT2 seed material, R26323-032-1, the experiment in Ethyl Acetate was repeated and seeded with the aforementioned lot. The resulting isolated material, R26323-032-3, appeared to be exclusively CSPNCT2 co-crystal, with no presence of CSP-1103 or NCT.

The following conclusions were drawn. Co-crystal can be formed readily in DCM or Ethyl Acetate by using a seeding technique with co-crystal template. Using a mixed polymorph co-crystal seed in DCM appears to lead to exclusively CSPNCT2, whereas in Ethyl Acetate it leads to a mixture of forms (CSPNCT1 and CSPNCT2) like its seed template. When seeding in Ethyl Acetate with exclusively CSPNCT2, the resulting isolated material is also CSPNCT2.

### EXAMPLE 4

### Synthesis of CSPNCT2 Co-Crystal

CSPNCT2 Co-Crystal was synthesized from either EtOAc or DCM.

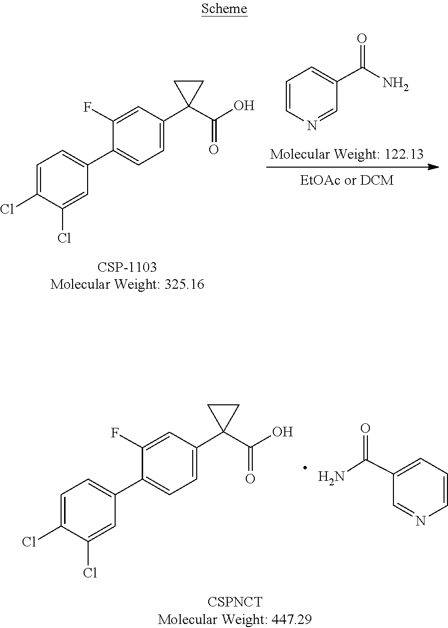 [SMILES: O=C(O)C1(c2ccc(-c3ccc(Cl)c(Cl)c3)c(F)c2)CC1 ; NC(=O)c1cccnc1 ; O=C(O)C1(c2ccc(-c3ccc(Cl)c(Cl)c3)c(F)c2)CC1 ; NC(=O)c1cccnc1]

An expected yield for synthesis from DCM was 49%, and an expected yield from EtOAc was 59%.

### Synthesis from DCM

TABLE 5
Table of Materials for CSPNCT2 Formation in DCM

| | | | Multi- | | | |
|---|---|---|---|---|---|---|
| | | | plier | | | |
| Reagent | MW | mmol | (g/g) | Input | | Lot # |
| CSP-1103 | 325.16 | 12.3 | 1.00 | 4.0 | g | 82398 |
| Nicotinamide | 122.13 | 12.3 | 0.375 | 1.5 | g | 81938 |
| (NCT) | | | | | | |
| DCM (Initial | 84.93 | 1250 | 26.5 | 106.0 | g | 71600 |
| Charge) | | | | | | |
| DCM (NCT | 84.93 | 314 | 6.65 | 26.6 | g | 71600 |
| rinse) | | | | | | |
| DCM (Product | 84.93 | 157 | 3.325 | 13.3 | g | 71600 |
| wash) | | | | | | |
| CSPNCT2 Co- | 447.29 | 0.045 | 0.005 | 0.020 | g | R26323- |
| Crystal (Seed) | | | | | | 032-1 |

### Experimental

CSP-1103 (1.00 g/g) and DCM (26.5 g/g) were charged to vessel, agitated and heated to 40° C. Then, nicotinamide was charged as a solid (0.375 g/g) and rinsed in with DCM (6.65 g/g). The resulting mixture was heated back to 40° C. and stired until a clear solution resulted. Heat was removed and the solution was cooled to 30° C. Then, seed crystal (0.005 g/g) was charged. The solution was cooled to 20-25° C. while stirring, to precipitate solids. The solution was held at 20-25° C. for 1 hour and 20 minutes. The solids were then filtered, and the cake was washed with DCM (3.325 g/g). Then, the solids were dried for 24 hours at full vacuum and 20-25° C. to afford CSPNCT2.

The appearance of CSPNCT2 was white solid.

The yield of the synthesis was determined to be 49%.

### Synthesis from EtOAc

TABLE 6
Table of Materials for CSPNCT2 Formation in EtOAc

| | | | Multiplier | | | |
|---|---|---|---|---|---|---|
| Reagent | MW | mmol | (g/g) | Input | | Lot # |
| CSP-1103 | 325.16 | 15.4 | 1.00 | 5.0 | g | 82398 |
| Nicotinamide | 122.13 | 15.4 | 0.375 | 1.9 | g | 81938 |
| (NCT) | | | | | | |
| EtOAc (Initial | 88.11 | 512 | 9.02 | 45.1 | g | 80322 |
| Charge) | | | | | | |
| EtOAc (NCT | 88.11 | 103 | 1.82 | 9.1 | g | 80322 |
| rinse) | | | | | | |
| EtOAc (Product | 88.11 | 103 | 1.82 | 9.1 | g | 80322 |
| wash) | | | | | | |
| CSPNCT2 Co- | 447.29 | 0.056 | 0.005 | 0.025 | g | R26323- |
| Crystal (Seed) | | | | | | 032-1 |

### Experimental

CSP-1103 (1.00 g/g) and Ethyl Acetate (9.02 g/g) were charged to vessel, agitated and heated to 40° C. Then, Nicotinamide was charged as a solid (0.375 g/g) and rinsed in with Ethyl Acetate (1.82 g/g). The resulting mixture was heated back to 40° C. and stirred until a clear solution resulted. Heat was removed and the solution was cooled to 30° C. Then, seed crystal (0.005 g/g) was charged, and the solution was cooled to 20-25° C. while stirring, to precipitate solids. The solution was held at 20-25° C. for 1 hour and 15 minutes. The solids were filtered, and cake was washed with Ethyl Acetate (1.82 g/g). The solids were dried for at 27 hours and 30 minutes at full vacuum and 20-25° C. to afford CSPNCT2.

The appearance of CSPNCT2 was white solid.

The yield of the synthesis was determined to be 59%.

### EXAMPLE 5

CSPNCT2 and CSPNCT1 were analyzed by XRPD. The peaks of the XRPD pattern of CSPNCT2 are provided in Table 8, and the peaks of the XRPD pattern of CSPNCT1 are provided in Table 9.

TABLE 8

| Caption (display) | FWHM | Rel. Intensity |
|---|---|---|
| 17.779° | 0.178 | 100.0% |
| 5.736° | 0.195 | 22.2% |
| 18.175° | 0.186 | 12.6% |
| 17.076° | 0.188 | 12.1% |
| 36.959° | 0.206 | 11.7% |
| 28.572° | 0.197 | 10.4% |
| 14.152° | 0.224 | 8.2% |
| 31.488° | 0.185 | 7.3% |
| 22.797° | 0.179 | 6.5% |
| 13.935° | 0.160 | 3.7% |
| 25.664° | 0.189 | 3.6% |
| 35.907° | 0.194 | 2.9% |
| 22.291° | 0.158 | 2.4% |
| 19.680° | 0.177 | 2.3% |
| 29.016° | 0.174 | 2.2% |
| 37.389° | 0.199 | 2.2% |
| 39.757° | 0.187 | 2.2% |
| 15.139° | 0.170 | 2.2% |
| 29.487° | 0.229 | 2.1% |
| 20.136° | 0.166 | 2.0% |
| 11.356° | 0.153 | 2.0% |
| 24.078° | 0.162 | 1.7% |
| 8.530° | 0.120 | 1.7% |
| 16.495° | 0.153 | 1.3% |
| 33.216° | 0.152 | 1.0% |

TABLE 9

| Caption (display) | FWHM | Rel. Intensity |
|---|---|---|
| 20.025° | 0.181 | 100.0% |
| 18.462° | 0.160 | 65.9% |
| 18.243° | 0.160 | 54.4% |
| 14.630° | 0.141 | 39.3% |
| 16.706° | 0.202 | 32.5% |
| 14.901° | 0.197 | 31.8% |
| 22.014° | 0.206 | 30.8% |
| 24.173° | 0.185 | 20.6% |
| 26.145° | 0.126 | 20.4% |
| 29.975° | 0.196 | 20.2% |
| 26.136° | 0.126 | 18.9% |
| 20.267° | 0.160 | 18.1% |
| 34.062° | 0.186 | 16.1% |
| 22.271° | 0.160 | 15.7% |
| 15.561° | 0.161 | 13.6% |
| 26.457° | 0.224 | 13.4% |
| 24.467° | 0.176 | 12.9% |
| 30.636° | 0.197 | 10.7% |
| 28.853° | 0.201 | 9.8% |
| 27.828° | 0.246 | 8.8% |
| 29.374° | 0.198 | 6.8% |
| 36.813° | 0.233 | 6.2% |
| 36.476° | 0.292 | 6.2% |
| 19.034° | 0.177 | 5.3% |
| 32.419° | 0.108 | 5.2% |
| 7.450° | 0.216 | 4.4% |
| 31.639° | 0.180 | 4.3% |
| 39.140° | 0.249 | 4.2% |
| 13.859° | 0.162 | 4.1% |
| 21.647° | 0.230 | 4.0% |
| 19.395° | 0.149 | 3.7% |
| 11.158° | 0.204 | 3.6% |
| 25.096° | 0.269 | 3.0% |
| 23.530° | 0.200 | 3.0% |
| 36.005° | 0.215 | 2.4% |
| 35.650° | 0.251 | 1.9% |
| 35.191° | 0.156 | 1.4% |

### EXAMPLE 6

### Crystallization Scale Up

In order to test the reproducibility and scalability of co-crystal formation using a seeding technique, the process was run at gram scale in both DCM and Ethyl Acetate. With success from both experiments, the procedures are described above in Example 4. Both experiments were seeded with, a pure CSPNCT2 co-crystal (R26323-032-1).

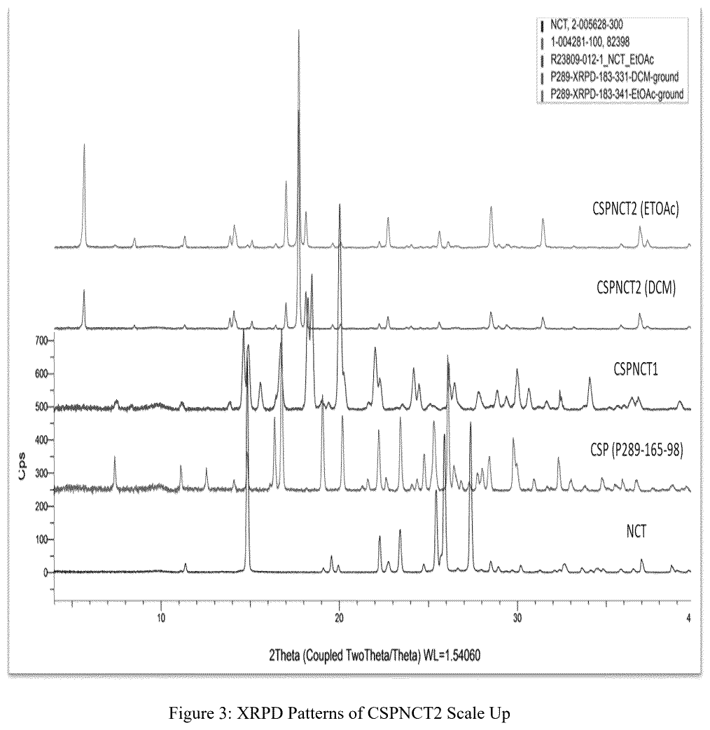

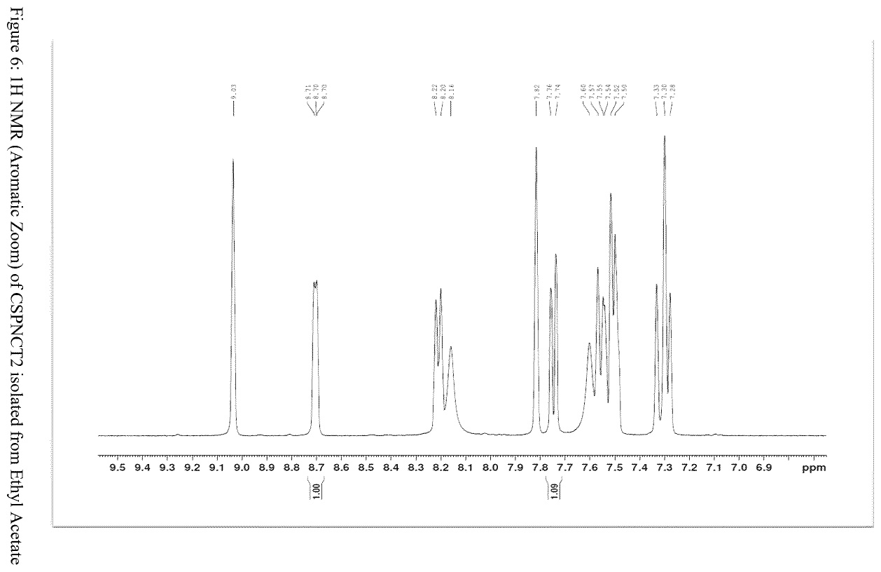

R26323-033-1, a material isolated from DCM, was a white fluffy solid with static like properties. 2.7 g of solid was isolated from a 4.0 g experiment, consistent with a 49% recovery based on theoretical yield. The XRPD pattern (FIG. 3) was consistent with CSPNCT2 co-crystal and there was no presence of either individual co-former. 1H NMR showed a 1:1 ratio of CSP-1103 and NCT, indicating a 1:1 co-crystal. This can be observed in FIG. 5 where an NCT doublet at 8.7 ppm is integrated at 1.0 compared to the CSP-1103 doublet at 7.7 ppm at 1.06.

R26323-034-1, material isolated from EtOAc, was solid with similar properties to the material isolated from DCM. 4.1 g of solid was isolated from a 5.0 g experiment, consistent with a 59% recovery based on theoretical yield. The physical properties of the solid were similar to the CSPNCT2 generated in DCM.

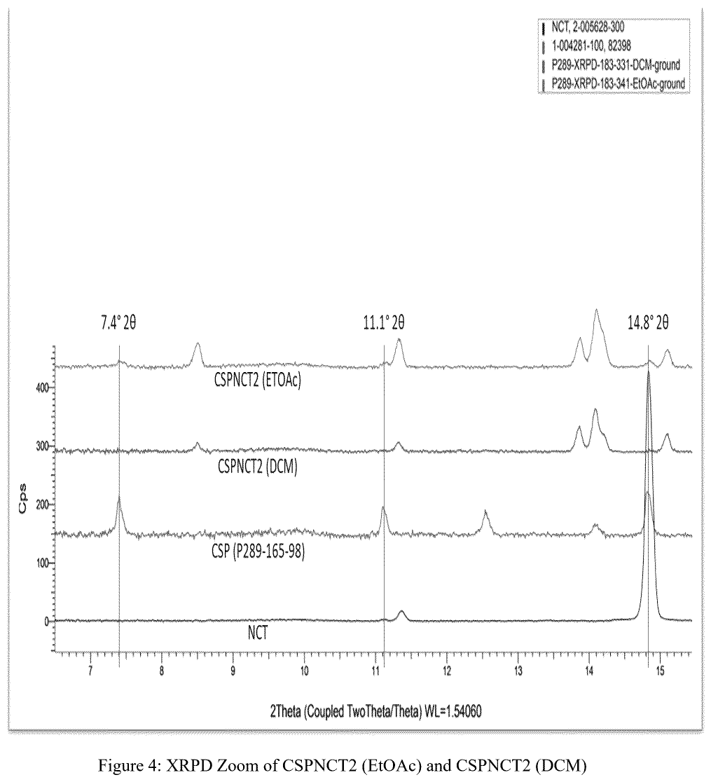

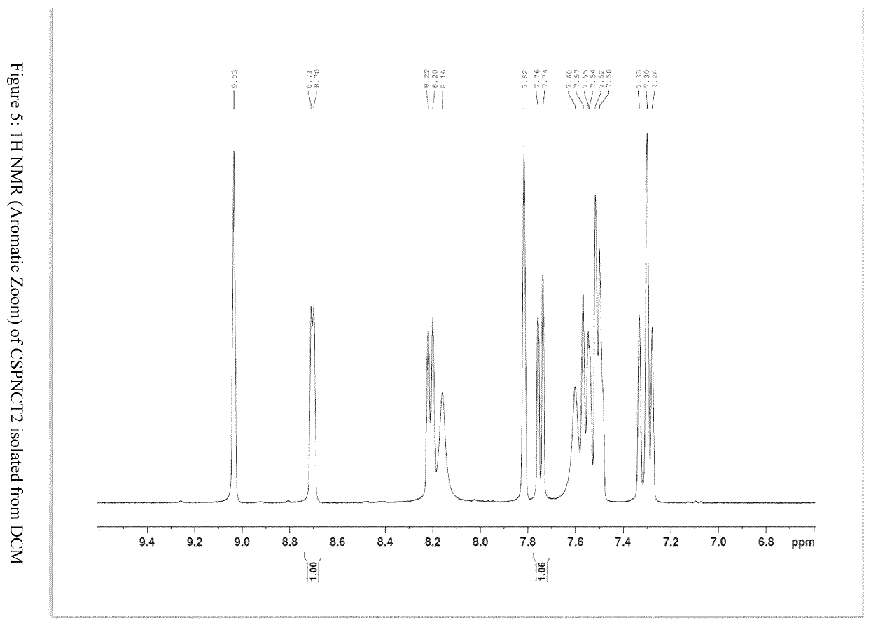

The XRPD pattern was consistent with CSPNCT2 co-crystal although there was a minimal presence of CSP-1103. This can be observed in FIG. 3. The green trace shows the XRPD pattern of R26323-034-1 compared to other traces. In FIG. 4, a zoom of the Ethyl Acetate and DCM traces compared to CSP-1103 shows the trace response of CSP-1103 in the CSPNCT2 material generated in Ethyl Acetate. Though there is potentially indicating trace amounts of CSP-1103 present, 1H NMR showed an approximate 1:1 ratio of CSP-1103 and NCT, indicating a 1:1 co-crystal. This can be observed in FIG. 6 where an NCT doublet at 8.7 ppm is integrated at 1.0 compared to the CSP-1103 doublet at 7.7 ppm at 1.09.

Both experiments reveal two processes that appear to scale consistently to obtain CSPNCT2. The experiments also show that seed material can be propagated with each subsequent experiment and used as a seed for another scale up.

TABLE 7
Crystallization Scale Up Reactions

| | | Input | Re- | | NMR |
|---|---|---|---|---|---|
| | | (CSP- | covery | XRPD co- | (CSP- |
| Pilot | Lot # | 1103) | (%) | crystal | 1103:NCT) |
| DCM - | R26323- | 4.0 g | 2.7 g | CSPNCT2 | 1:1 |
| Seeded with | 033-1 | | (49%) | | |
| R26323-032-1 | | | | | |
| Ethyl Acetate- | R26323- | 5.0 g | 4.1 g | CSPNCT2 | 1:1 |
| Seeded | 034-1 | | (59%) | | |
| R26323-032-1 | | | | | |

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

What is claimed is:

1. A polymorph of a co-crystal of itanapraced and nicotinamide, the polymorph comprising a crystal lattice comprising itanapraced and nicotinamide in a stoichiometric ratio of about 1:1, wherein the polymorph has an X-ray Powder Diffraction Pattern (XRPD) having at least five peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering).

2. The polymorph of claim 1, wherein the polymorph has an X-ray Powder Diffraction Pattern (XRPD) that is substantially the same as the XRPD shown in FIG. 8.

3. The polymorph of claim 1, wherein the itanapraced and the nicotinamide are not bound covalently and are not ionically bound in the polymorph.

4. A crystalline system comprising itanapraced and nicotinamide in a crystal lattice, wherein the crystalline system has an X-ray Powder Diffraction Pattern (XRPD) having at least five peaks at positions selected from the group consisting of 17.8°, 5.7°, 18.2°, 17.1°, 37.0°, 28.6°, 14.2°, 31.5°, 22.8°, 13.9°, 25.7°, 35.9°, 22.3°, 19.7°, 29.0°, 37.4°, 39.8°, 15.1°, 29.5°, 20.1°, 11.4°, 24.1°, 8.5°, 16.5°, and 33.2°, expressed in 2θ, produced from a Cu radiation source (λ=1.54 Å after Ni filtering).

5. A crystalline system comprising itanapraced and nicotinamide in a crystal lattice and in a stoichiometric ratio of about 1:1, wherein itanapraced and nicotinamide are linked by hydrogen bonding and other non-covalent and non-ionic interactions, the crystalline system is solid at 25° C. and has an X-ray Powder Diffraction Pattern (XRPD) that is substantially the same as the XRPD shown in FIG. 8.

6. A method of synthesizing a polymorph of a co-crystal of itanapraced and nicotinamide, the method comprising dissolving itanapraced and nicotinamide in dichloromethane to form a solution and precipitating the polymorph of the co-crystal of itanapraced and nicotinamide from the solution.

7. The method of claim 6, further comprising heating the solution from to a temperature between about 30° C. and about 50° C.

8. The method of claim 6, further comprising cooling the solution from the temperature between about 30° C. and about 50° C. to a temperature between about 15° C. and about 40° C.

9. The method of claim 6, wherein the solution is held at 20-25° C. for no less than 1 hour.

10. The method of claim 9, wherein the solution is held at 20-25° C. for 1 to 24 hours.

11. The method of claim 8, wherein the method further comprises adding a seed crystal to the solution prior to cooling.

12. A method of synthesizing a polymorph of a co-crystal of itanapraced and nicotinamide, the method comprising dissolving itanapraced and nicotinamide in ethyl acetate to form a solution, adding a seed crystal to the solution and precipitating the polymorph of the co-crystal of itanapraced and nicotinamide from the solution.

13. The method of claim 11 or claim 12, wherein the seed crystal is the polymorph of claim 1, the polymorph of claim 2 or the polymorph of claim 3.

14. The method of claim 12, wherein the seed crystal further comprises an additional polymorph of itanapraced and nicotinamide.

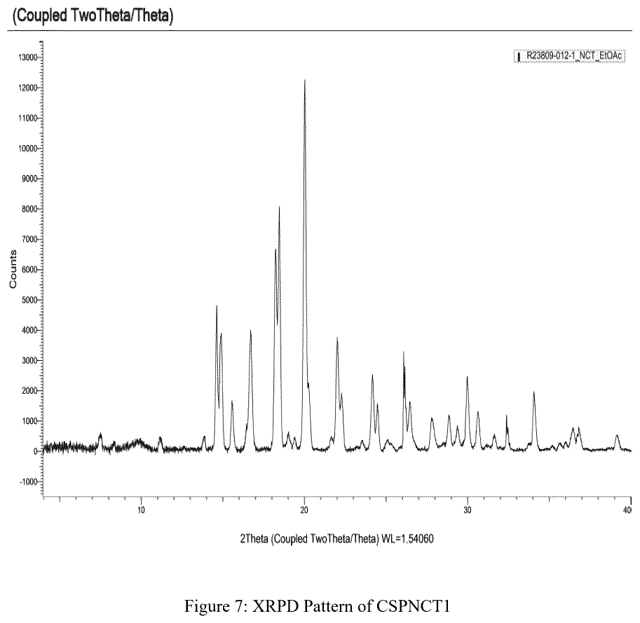

15. The method of claim 14, wherein the additional polymorph has an X-ray Powder Diffraction Pattern (XRPD) that is substantially the same as the XRPD shown in FIG. 7.

16. A pharmaceutical composition comprising a mixture comprising (i) the polymorph of claim 1, the polymorph of claim 2 or the polymorph of claim 3 and (ii) an additional polymorph of itanapraced and nicotinamide.

17. The pharmaceutical composition of claim 16, wherein the additional polymorph has an X-ray Powder Diffraction Pattern (XRPD) that is substantially the same as the XRPD shown in FIG. 7.

18. A pharmaceutical dosage form comprising (i) the polymorph of claim 1, the polymorph of claim 2 or the polymorph of claim 3 and (ii) and one or more pharmaceutically acceptable excipients, wherein the polymorph of claim 1, the polymorph of claim 2 or the polymorph of claim 3 comprises from about 1% to about 99% of the pharmaceutical dosage form by weight.

19. The pharmaceutical dosage form of claim 18, wherein the pharmaceutical dosage form further comprises an additional polymorph of itanapraced and nicotinamide, and the additional polymorph has an X-ray Powder Diffraction Pattern (XRPD) that is substantially the same as the XRPD shown in FIG. 7.

20. A method of treating a neurodegeneration disorder in a subject in need thereof comprising administering to the subject a therapeutically effective amount of the polymorph of claim 1, the polymorph of claim 2, the polymorph of claim 3, the crystalline system of claim 4, or the crystalline system of claim 5.
